(19) United States
(12) Patent Application Publication
(10) Pub. No.: US 2021/0291182 A1
(43) Pub. Date: Sep. 23, 2021

(54) Multiplex Hybrid Microfluidics Analyte Detection Systems

(71) Applicant: BRISA Biotech, LLC, College Park, MD (US)

(72) Inventors: Kenneth John Gollob, College Park, MD (US); Samuel Dutra Gollob, College Park, MD (US)

(73) Assignee: BRISA Biotech, LLC, College Park, MD (US)

(21) Appl. No.: 16/461,480

(22) PCT Filed: Nov. 16, 2017
(86) PCT No.: PCT/US17/62094

§ 371 Date: May 16, 2019

Related U.S. Application Data
(60) Provisional application No. 62/423,545, filed on Nov. 17, 2016.

(51) Int. Cl. B01L 3/00; G01N 33/543

(52) U.S. Cl. CPC B01L 3/502746; B01L 3/502715; B01L 3/5023; G01N 33/54366; B01L 2300/0864; B01L 2400/0406; B01L 2200/16; B01L 2200/027; B01L 2400/0487; B01L 2300/0681; B01L 2300/087

(57) ABSTRACT

Embodiments of the present invention relate to point-of-care systems for analyte detection, and more particularly, to systems and methods for detecting analytes in a fluid by passing the analytes through one or more processing chambers in a pressure driven process, and subjecting the processed analytes for detection in a lateral flow process.

### FIELD OF THE INVENTION

The present disclosure relates generally to point-of-care systems for analyte detection, and more particularly, to systems and methods for detecting analytes in a fluid by passing the analytes through one or more distinct processing chambers in a pressure driven process, followed by subjecting the processed analytes for detection in a lateral flow process.

### BACKGROUND

Conventional automated fluidic systems are generally, complex systems with multiple components and modules, frequently relying on computerized systems for control. Such systems may be used to analyze nucleic acids and/or perform sequencing, sort cells as well as identify chemical analytes. These systems also frequently employ sophisticated methods of detection based on magnetic, optical, or spectrographic techniques (see, e.g., McNerney R., Diagnostics for Developing Countries, Diagnostics (2015) 5: 200-209. Available from: http://www.mdpi.com/2075-4418/5/2/200/htm, Choi S., Powering point-of-care diagnostic devices, Biotechnol Adv. (2016) 34: 321-330. Available from: http://dx.doi,org/10.1016/j.biotechadv.2015.11,004; Jung W. et al., Point-of-care testing (POCT) diagnostic systems using microfluidic lab-on-a-chip technologies. Microelectron Eng. (2014) 132: 46-57. Available from: http://dx.doi.org/10.1016/j.mee.2014.09.024). Accordingly, such systems can be difficult to set up, to maintain, and costly to operate. Moreover, many of these systems are geared toward analyzing one analyte at a time.

For medical and other health care practitioners that are practicing in remote areas or are in areas without access to such automated systems, obtaining fast, accurate test results of whether a patient has been infected with a particular pathogen, or afflicted with a specific disease, is not feasible with these complex systems. Moreover, for health officials seeking to monitor the spread of an infectious agent in real-time or near real-time is not possible, as the samples would need to be obtained from a patient, sent to a laboratory for analysis, placed in a queue for analysis and analyzed, and once analyzed, the results would then be send back to the practitioner. Such a process often takes a week or longer, during which time, the transmissible infection may have significantly spread, making quarantine difficult or impossible. In addition, application and reading of complex diagnostic tests often requires specialized training, however, in remote areas and during disease outbreaks, it is often necessary to rely on non-medical personnel for the application and reading of a diagnostic test.

In recent years, numerous transmissible diseases that have first been detected in a particular geographic location have spread beyond their original boundaries to emerge as global health threats. For example, mosquito-borne diseases such as Zika, Dengue fever, Chikungunya and other infectious diseases continue to spread globally and are now present on nearly all continents. Importantly, due to overlapping clinical symptoms, and complex diagnostic algorithms involving both detection of the pathogen and detection of the host response against the pathogen, the need for an easy to use multiplex diagnostic device is clear. Other diseases such as Ebola and influenza have had outbreaks that have been difficult and costly to control.

Medical practitioners in the field need rapid point-of-care diagnostic tests that can test for a variety of diseases in order to correctly diagnose patients in a timely manner, in order to deliver effective and life-saving treatment to infected patients, as well as contain the spread of the disease by enacting quarantines. Moreover, in the case of deadly rapidly progressing diseases (e.g., some forms of acute myeloid leukemia), medical practitioners treating patients in remote areas of developed countries and in many areas of underdeveloped countries, also need a rapid point-of-care diagnostic test that can detect these diseases.

Thus, what is needed are new, cost effective, rapid point-of care devices that operate independently of advanced computerized systems, that can be applied and read by nonskilled personnel, and can quickly diagnose a plurality of diseases from a biological sample

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Embodiments of the present invention are directed towards a point-of-care system comprising an inlet chamber for introducing a fluid comprising analytes into the device; a first fluid flow pathway in fluid communication with the inlet chamber, wherein the first fluid flow pathway is configured to process the analytes according to a first process; a second fluid flow pathway in fluid communication with the inlet chamber, wherein the second fluid flow pathway is configured to process the analytes according to a second process that is different from the first process; a first channel in fluid communication with the first fluid flow pathway, wherein the first channel comprises one or more readout bands configured to hind to the analytes processed by the first fluid flow pathway; a second channel in fluid communication with the second fluid flow pathway, wherein the second channel comprises one or more readout bands configured to bind to the analytes processed by the second fluid flow pathway.

Generally, the invention is directed to a pressure driven fluidics device comprising (a) an inlet for introducing by manually applied pressure a fluid comprising an analyte into the device; (b) at least two fluid flow pathways in fluid communication with the inlet, wherein each fluid flow pathway comprises one or more chambers, wherein each chamber is configured to perform one or more different processing steps to process the analyte and wherein each fluid flow pathway comprises at least one flow regulation tube; and (c) at least one lateral flow strip in fluid communication with each fluid flow pathways, wherein the lateral flow strip comprises one or more readout bands configured to detect the differently processed analyte. In some embodiments, a filter can be positioned between the inlet and the fluid flow pathway.

The processing chambers of the device can perform a number of functions, including, but not limited to (a) a denaturing chamber comprising one or more denaturing reagents to denature the analyte; (b) a neutralization chamber comprising one or more neutralization reagents to neutralize the analyte; (c) a solubilizing chamber comprising one or more soluhilizing reagents to solubilize the analyte; (d) a fragmentation chamber comprising one or more fragmenting or cleavage reagents; (e) an impurity removal chamber comprising one or more reagents to remove one or more non-analyte molecules from the fluid; (f) a lysis chamber comprising one or more reagents to lyse cells; (g) a precipitation chamber comprising one or more reagents to precipitate the analyte; (h) a deglycosylation chamber comprising one or more reagents to deglycosylate the analyte; (i) a sample pre-treatment zone for removal of one or more unwanted or competitor molecules from the fluid; (j) both (f) and (g); (k) both (a) and (b); (l) both (f) and (h); (m) each of (a), (d) and (h); (n) each of (a), (b), (d) and optionally (h); or (m) any combination of (a)-(n).

The diameter and/or curvature of the flow regulation tube can be selected based upon the viscosity of the fluid and/or the desired flow rate. For example, the diameter of the flow regulation tub is selected from: (a) 1-3 mm, (b) 1-4 mm, (c) 1-5 mm, (d) 2-4 mm, (e) 2-5 mm, (f) 3-5 mm, (g) 4-5 mm or (h) 1-5 mm. In preferred embodiments, the fluid flows through the lateral flow strip by capillary action and/or the analyte can be further processed on the lateral flow strip prior to contacting the readout bands.

In other embodiments, the fluid can be (a) mixed with an additional buffer, preferably EDTA or heparin, before introduced into the device; (b) introduced into the device by a collection tube inserted into the inlet; (c) introduced into the device by a syringe; (d) any combination of (a)-(d).

Fluids that can be applied to the device, include, but are not limited to blood, saliva, urine, feces, an environmental sample, or a biological sample comprising a pathogen or molecule(s) associated with the presence of a disease. Additionally, the analyte that is processed and/or detected by the device, includes, but is not limited to a lipid, a carbohydrate, a protein, an organic compound, an inorganic compound, a nucleic acid, or a cancer specific antigen. In preferred embodiments, the analyte is an antibody.

In further preferred embodiments, the first flow pathway is capable of detecting a first analyte comprising a protein antigen and the second flow pathway is capable of detecting a second analyte comprising an antibody that binds to the protein antigen. The protein antigen can be found, for example, in solution in the fluid or intracellularly (which is released from the cell by one of the processing steps—i.e., by the denaturing, lysis, and/or fragmentation chamber.)

In further preferred embodiments, the device comprises multiple fluid flow pathways configured to detect and distinguish between Dengue infection, Zika infection and Chikungunya infection. For example, the device could be configured such that: (a) at least one fluid flow pathway capable of detecting any one of Dengue, Zika or Chikungunya extracellular antigens in the fluid; (b) at least one fluid flow pathway capable of detecting any one of Dengue, Zika or Chikungunya intracellular antigen in the fluid; (c) at least one fluid flow pathway capable of detecting either a IgM or IgG antibody that can bind to any one of Dengue, Zika or Chikungunya antigen, removing Dengue false positives; (d) at least one fluid flow pathway capable of detecting either a IgM or IgG antibody that can bind to any one of Dengue, Zika or Chikungunya antigen, removing Zika false positives; and (e) at least one fluid flow pathway capable of detecting a IgM or IgG antibody that can bind to any one of Dengue, Zika or Chikungunya antigen, removing Chikungunya false positives.

In further preferred embodiments, the detection of the analyte by the first and/or second readout bands indicates whether a patient has an active or past infection, a cancer/tumor, an allergy/allergic immune response, or a non-communicable disease. For example, (a) the infection could be a Mosquito borne illnesses, including but are not limited to malaria, dengue fever, West Nile virus, chikungunya, yellow fever, filariasis, Japanese encephalitis, Saint Louis encephalitis, Western equine encephalitis, Eastern equine encephalitis, Venezuelan equine encephalitis, La Crosse encephalitis and Zika fever; (b) the tumor antigen could be MAGE-A1, MAGE-A2, MAGE-A3, MAGE-A4, MAGE-A5, MAGE-A6, MAGE-A7, MAGE-A8, MAGE-A9,MAGE-A10, MAGE-A11, MAGE-A12, GAGE-1, GAGE-2, GAGE-3, GAGE-4, GAGE-5, GAGE-6, GAGE-7, GAGE-8, GAGE-9, BAGE, BAGE-1, RAGE, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, MAGE-C3, MAGE-C4, MAGE-C5, NY-ESO-I, LAGE-I, SSX-1, SSX-2 (1-HOM-MEL-40), SSX-3, SSX-4, SSX-5, SCP-I and XAGE, melanocyte differentiation antigens, p53, ras, p21ras, CEA, MUCI, PMSA, PSA, tyrosinase, Melan-A, MART-1, gp100, gp75, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-2, and 3, neo-PAP, myosin class I, OS-9, pml-RAR alpha fusion protein, PTPRK, K-ras, N-ras, Triosephosphate isomerase, Gnt-V, Herv-K-mel, NA-88, SP17, and TRP2-Int2, MART-1, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, alpha.-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\170K, NY-CO-1, RCAS1, SDCCAG16, TA-90 (Mac-2 binding protein\cyclophilin C-associated protein), TAAL6, TAG72, TLP, TPS, tyrosinase related proteins, TRP-1, TRP-2, TRP-2/INT2, or mesothelin, HER-2/neu, 707-A, AFP, ART-4, p190 minor bcr-abl, CAMEL, CAP-1, CAP-2, CDC27, CT, Cyp-B, DAM, ELF2M, GAGE, HAGE, HLA-A*0201, HLA-A*1101, HLA-A*0201-R1701, HLA-B*0702, HPV-E7, HAST-2, hTERT (or hTRT), iCE, LAGE, LDLR/FUT, MC1R, myosine/m, MUC1, NA88-A, NY-ESO-1, PRAME, PSA-1, PSA-2, PSA-3, prostate-specific membrane antigen (PSMA), PSM, RU1 or RU2, SAGE, SART-1 or SART-3, TEL/AML1, TPI/m, and WT1, adenosine deaminase-binding protein (ADAbp), FAP, cyclophlin b, CRC C017-1A/GA733, AML1, CD20, alpha-fetoprotein, E-cadherin, alpha-catenin, beta-catenin, gamma-catenin, p120ctn, adenomatous polyposis coli protein (APC), fodrin, Connexin 37, Ig-idiotype, p15, GM2 ganglioside, GD2 ganglioside, Smad family of tumor antigens, Imp-1, PIA, EBV-encoded nuclear antigen (EBNA)-1, brain glycogen phosphorylase, CT-7, and c-erhB-2; (c) the infection could be one of a tick-borne illness, including but not limited to Anaplasmosis, Babesiosis, Borrelia mayonii, Borrelia miyamotoi, Colorado tick fever, Ehrlichiosis, Heartland virus, Lyme disease, Powassan disease, Rickettsia parkeri rickettsiosis, Rocky mountain spotted fever (RMSF), Southern tick-associated rash illness (STARI), Tickborne relapsing fever (TBRF), Tularemia, and 364D rickettsiosis; (d) the non-communicable disease could be a cardiovascular disease (e.g., coronary heart disease, stroke, etc.), cancer, chronic respiratory disease, diabetes, chronic neurologic disorders (e.g., Alzheimer's disease, dementias), and arthritis/musculoskeletal diseases; (e) the infection could be a bacterial illness, including but not limited to Streptococcal bacteria, Escherichia coli, Salmonella, Helicobacter pylori, Neisseria gonorrhoeae, Neisseria meningitides, Staphylococcus aureus, Streptococcal bacteria, Actinomycoses and nocardiosis, Anthrax, Brucellosis, Buruli ulcer, Capnocytophaga, Elizabethkingia, Glanders (Burkholderia mallei). Hansen's disease (Leprosy), Leptospirosis, Melioidosis (Burkholderia pseudomallei), Pasteurella sp. infections, and Rat-Bite fever; (f) the infection could also be a parasitic illness, including but not limited to protozoan (e.g., leishmaniasis, chagas disease, malaria, toxoplasmosis, etc.) and helminths (e.g., tapeworms, flukes, roundworms, etc.); (g) the infection could also be a fungal illness, including but not limited to Aspergillosis, Blastomycosis, Candidiasis, Coccidioidomycosis, C. neoformans infection, C. gattii. Infection, Fungal Eye Infections, Fungal Nail Infections, Histoplasmosis, Mucormycosis, Pneumocystis pneumonia, Ringworm, and Sporotrichosis; (h) the allergy could be a food preferably, peanuts, tree nuts, crustacean shellfish, fish, wheat (gluten), milk, egg, soybeans and/or airborne allergies, preferably pollen, mold, dust mite, and/or animals; (i) the infection could be HIV; and/or (j) the cancer could be a leukemia, adenocarcinoma, sarcoma, skin cancer, melanoma, bladder cancer, brain cancer, breast cancer, uterine cancer, ovarian cancer, prostate cancer, lung cancer, colorectal cancer, cervical cancer, liver cancer, head and neck cancer, esophageal cancer, pancreas cancer, pancreatic ductal adenocarcinoma (FDA), renal cancer, stomach cancer, multiple myeloma and/or cerebral cancer. The device could be used to detect whether a patient has an active or past infection, a cancer/tumor, an allergy/allergic immune response, a non-communicable disease; and/or any of (a)-(j).

Additionally, embodiments of the present invention include methods of detecting analytes using the pressure driven fluidics device comprising: introducing a fluid comprising analytes into the device via an inlet; applying pressure, e.g., using a syringe attached to the inlet, to cause the fluid to flow through the first fluid flow pathway and the second fluid flow pathway; and using capillary action to cause the fluid from the first fluid flow pathway to flow through one or more first channels configured to bind to the analytes processed by the first process, and using capillary action to cause the fluid from the second fluid flow pathway to flow through one or more second channels configured to hind to the analytes processed by the second process. The analytes are then detected, by binding to a particular reagent, in the first or second channels.

In some embodiments, the fluid comprising the analytes flows through the first fluid flow pathway and through the second fluid flow pathway by manually applied pressure, and wherein fluid flows through the first channel and through the second channel by capillary action.

In preferred embodiments, the inlet chamber connects to the first fluid flow pathway and to the second fluid flow pathway, wherein the first fluid flow pathway and the second fluid flow pathway are not directly connected to each other. For example, the first fluid flow pathway and the second fluid flow pathway may diverge or branch from the inlet chamber to form separate pathways.

The first fluid flow pathway comprises one or more processing chambers. For example, in some embodiments, the first fluid flow pathway comprises a denaturing chamber in fluid communication with a neutralization chamber, with the denaturing chamber comprising one or more denaturing reagents to denature the analyte and the neutralization chamber comprising one or more reagents to neutralize the denaturing reagents. In other embodiments, the first fluid flow pathway comprises a denaturation/lysis chamber in fluid communication with a neutralization chamber, with the denaturation/lysis chamber comprising one or more reagents to denature and lyse cells in order to release the analyte into the fluid, and the neutralization chamber comprising one or more neutralization reagents to neutralize the denaturation/lysis reagents. In other embodiments, the first fluid flow pathway comprises a lysis chamber in fluid communication with a fragmentation chamber, with the lysis chamber comprising one or more reagents to lyse cells, and the fragmentation chamber comprising one or more fragmentation reagents to fragment the analyte.

In additional embodiments, the first fluid flow pathway comprises a lysis chamber in fluid communication with a denaturation chamber, and a denaturation chamber in fluid communication with a neutralization chamber, with the lysis chamber comprising one or more reagents to lyse cells to release the analyte, the denaturation chamber comprising one or more denaturation reagents to denature the analyte, and the neutralization chamber comprising one or more neutralization reagents to neutralize the denaturation/lysis reagents.

In other embodiments, the first fluid flow pathway comprises a fragmentation chamber, with the fragmentation chamber comprising one or more reagents to fragment the analyte.

In other embodiments, the first fluid flow pathway comprises a solubilization chamber, with the solubilization chamber comprising one or more reagents to solubilize the analyte.

In other embodiments, the first fluid flow pathway comprises a lysis chamber in fluid communication with a precipitation chamber, with the lysis chamber comprising one or more reagents to lyse cells and the precipitation chamber comprising one or more reagents to precipitate the analyte.

In still other embodiments, the first fluid flow pathway comprises a deglycosylation chamber, with the deglycosylation chamber comprising one or more reagents to deglycosylate the analyte.

In other embodiments, the first fluid flow pathway comprises a lysis chamber in fluid communication with a deglycosylation chamber, with the lysis chamber comprising one or more reagents to lyse cells and the deglycosylation chamber comprising one or more reagents to deglycosylate the analyte.

In still other embodiments, the second fluid flow pathway comprises an impurity removal chamber, with the impurity removal chamber comprising one or more reagents to remove contaminants and other non-analyte molecules that may cause false positives or false negatives with regard to detection of the analyte.

In some embodiments, each of the one or more readout bands of the first channel are configured to indicate the presence of the analyte, wherein the analyte is denatured, fragmented, or deglycosylated.

In still other embodiments, each of the one or more readout bands of the second channel are configured to indicate the presence of a non-denatured or non-fragmented analyte or an analyte that has not been deglycosylated.

In still other embodiments, the fluid introduced into the inlet chamber comprises a blood sample, a saliva sample, a urine sample, or other biological sample comprising a pathogen. in still other embodiments, the analytes comprise lipids, carbohydrates, proteins, organic or inorganic compounds, nucleic acids, or cancer specific antigens.

In still other embodiments, flow regulation tubes are utilized to connect the components of the pressure driven component of the device, e.g., from the inlet chamber to first fluid flow pathway, from the inlet chamber to the second fluid flow pathway, to connect the one or more chambers of the first fluid flow to each other, to connect the first fluid flow pathway to the sample delivery array, and to connect the second fluid flow pathway to the sample delivery array. As specific examples, flow regulation tubes are used to connect the inlet chamber to the denaturation chamber, the denaturation chamber to the neutralization chamber, and the neutralization chamber to the first sample delivery array. As another example, the flow regulation tubes can connect the inlet to the impurity removal chamber, and the impurity removal chamber to the second sample delivery array. In still other embodiments, the diameter of the flow regulation tubes is selected based upon the viscosity of the fluid and/or the desired flow rate.

In preferred embodiments, the system or device is configured to determine whether a patient has an active infection, a past infection, a cancer/tumor, an allergy/allergic immune response, or a non-communicable disease by detecting the analytes.

In some embodiments, the first fluid flow pathway comprises at least one processing chamber. In further embodiments, the first fluid flow pathway comprises at least 2, 3, 4, 5, 6, 7, 8, 9, 10, 11, 12, 13, 14, 15, 16, 17, 18, 19 or 20 processing chambers. In other embodiments, the second fluid flow pathway comprises at least one processing chamber. In further embodiments, the second fluid flow pathway comprises at least 2, 3, 4, 5, 6, 7, 8, 9, 10, 11, 12, 13, 14, 15, 16, 17, 18, 19 or 20 processing chambers.

100341 In other embodiments, the first fluid flow pathway is in fluid communication with at least one channel. In still other embodiments, the first fluid flow pathway is in fluid communication with 2, 3, 4, 5, 6, 7, 8, 9, or 10 channels. In other embodiments, the second fluid flow pathway is in fluid communication with at least one channel. In still further embodiments, the second fluid flow pathway is in fluid communication with 2, 3, 4, 5, 6, 7, 8, 9, or 10 channels.

In preferred embodiments, fluid flow through the chambers is driven by manually applied pressure. Manually applied pressure is applied by a pump, such a manual pump, or any other device not driven by an external electrical power source capable of generating pressure, which is applied to the device to cause the analyte-containing fluid to flow through the one or more chambers of the first fluid flow pathway and the one or more chambers of the second fluid flow pathway.

In preferred embodiments, fluid flow through the channels is driven by laminar flow or capillary action. In some embodiments, the channel may comprise a wick, sample or conjugate pad or other suitable material for drawing in liquid to promote fluid flow through the channel by capillary action.

The following is intended to be exemplary, and in no way is intended to be limiting with regard to the embodiments of the invention. Many different embodiments are understood to fall within the scope of the disclosure presented herein.

### BRIEF DESCRIPTION OF THE DRAWINGS

Embodiments are illustrated by way of example and not limited to the figures of the accompanying drawings, in which like references indicate similar elements and in which:

FIG. 1 is an illustration of an exemplary microfluidics point-of-care analyte detection platform, showing the top casing, the bottom casing and a syringe, according to embodiments of the invention.

FIGS. 2A-B are illustrations of example syringes that can be connected to the microfluidics point-of-care analyte detection platform., according to embodiments of the invention.

FIG. 3 is an illustration of the top casing shown in FIG. 1, according to embodiments of the invention.

FIG. 4 is an illustration of the bottom casing as shown in FIG. 1, showing the pressure-driven component of the microfluidics point-of-care analyte detection platfbrm according to embodiments of the invention.

FIGS. 5A-C are illustrations of different types of chambers of the pressure-driven component of the microfluidics point-of-care analyte detection platform according to embodiments of the invention.

FIG. 6 is an illustration of the bottom casing as shown in FIG. 1, showing the lateral flow component of the microfluidics point-of-care analyte detection platform according to embodiments of the invention.

FIG. 7 is an illustration of a lateral flow strip capable of being used in the device described herein.

FIG. 8 is an overall schematic for potential application of the described device directed towards the simultaneous detection of Dengue, Zika and Chikungunya.

FIGS. 9A-B shows how processing as described herein can be used to remove false IgG anti-Zika positive signal from Dengue patient samples.

FIGS. 10A-B shows that IgM signal intensity is increased by processing as described herein.

FIGS. 11A-13 shows detection of NS1 in patient samples is dramatically increased by processing as described herein.

FIGS. 12A-B shows results obtained with the lateral flow strips.

### DETAILED DESCRIPTION

### A. Definitions

Unless defined otherwise, all technical and scientific terms used herein have the same meaning as commonly understood by those of ordinary skill in the art, such as in the arts of peptide chemistry, cell culture, nucleic acid chemistry, and biochemistry. Standard techniques are used for molecular biology, genetic and biochemical methods (see Sambrook et al., Molecular Cloning: A Laboratory Manual, 3^{rd} ed., 2001, Cold Spring Harbor Laboratory Press, Cold Spring Harbor, N.Y.; Ausubel et al., Short Protocols in Molecular Biology (1999) 4^{th} ed., John Wiley & Sons, Inc; Greenfield, E. A., Antibodies A Laboratory Manual, 2nd edition (2013), Cold Spring Harbor Laboratory Press, Cold Spring Harbor, N.Y.; Hermanson, G. T., Bioconjugate Techniques, 3rd Edition (2013) Academic Press, which are incorporated herein by reference.

In the present invention, the “microfluidics point-of-care analyte detection device” is used interchangeably with “fluidics device” or “on-board processing fluidics device” or “pressure driven fluidics device” which refers to a device designed to permit separate, but simultaneous processing of an analyte by passing the analyte through independent fluidic flow pathways composed of analyte processing chambers. Once processed, the analytes are delivered to lateral flow strips for detection. The device as described herein can be made of any material. For example, glass, metals, including, but not limited to stainless metals, silicon, plastic, polymers, metals, and/or ceramic materials can be used to form the casing and chambers of the device. For example, preferred examples of materials that can be used include, but are not limited to polymers, such as, polyethylene, polypropylene, polystyrene, polyester, polyester PLA and other biosorbable plastics, polycarbonate, polyvinyl chloride, polyethersulfone, polyacrylate (e.g., Acrylic, PMMA), hydrogel (e.g., acrylate), polysulfone, polyetheretherketone, thermoplastic elastomers (e.g., TPE, TPU), thermoset elastomers, silicone, poly-p-xylylene (e.g., Parylene), fluoropolymers, a metal, including, but not limited to stainless steel, cobalt-base alloys, titanium, titanium-base alloys, and/or shape memory alloy, and/or a ceramic material including, but are not limited to glass ceramics, calcium phosphate ceramics, and/or carbon-based ceramics).

In the present invention, a “channel” is defined as a passage directing the flow of a fluid. For example, a channel may be an enclosed hollow tube. The cross section of the tube may be of any suitable geometry as is known by those of skill in the art In one example the cross section is circular, oval, square, rectangular and/or irregularly shaped. The tube may have a constant cross-sectional area and/or it may be variable (e.g. it may constrict in certain areas and/or expand in others). The cross section of the channel may change shape along its length. In other examples, the channel may be a depression, gutter, groove and/or furrow. This depression may be shallow, deep, narrow and/or wide. According to embodiments of the present invention, the channel is used to house a lateral flow strip. The channel is configured to promote lateral flow of the analyte down the lateral flow strip by capillary action.

In the present invention, “circular” as applied to the cross section of a chamber or channel is defined as having a generally round shape. In some embodiments, circular means that the length of the diameter measured anywhere along the cross section of the chamber or channel is identical to that measured at any other point (i.e. it is perfectly circular). In other embodiments, circular means that the length of the diameter measured anywhere along the cross section of the chamber or channel is approximately identical to that measured at any other point (i.e. it is perfectly circular).

As used herein, the term “inlet” refers to an input port or other opening through which fluid is introduced into the pressure driven fluidics device. The term “inlet chamber” refers to a chamber that holds the fluid introduced into the device prior to processing by a fluid flow pathway.

As used herein, the term “outlet” refers to an output port or other opening through which fluid exits the pressure driven fluidics device.

As used herein, the term “analyte” refers to chemical substances, including but not limited to lipids, carbohydrates, proteins, organic or inorganic compounds, nucleic acids, natural or manufactured substances, or any other type of molecule obtained from a biological source, or any combination thereof, that are introduced into the pressure driven fluidics device described herein to undergo processing and detection by the pressure driven fluidics device.

Examples of lipids include, but are not limited to, fatty acids such as phospholipids, sterols, sphingolipids, inositols, mono-, di- and tri-glycerides, glycolipids, lipoproteins, etc. Examples of carbohydrates include, but are not limited, to mono-, di, and polysaccharides, glycoproteins, glycolipids, etc. Examples of proteins include, but are not limited to, simple proteins and conjugated proteins such as glycoproteins, lipoproteins, and nucleoproteins which include proteins such as: antibodies, structural proteins, extracellular or intracellular cell signaling molecules (cytokines, chemokines, kinases, STATs), enzymes, gas transport proteins, nutrient proteins, protein hormones, and proteins that perform mechanical work. Examples of organic compounds include, but are not limited to, lipids, proteins, vitamins, and carbohydrates. Examples of inorganic compounds include, but are not limited to, simple (metals and non-metals) and complex (oxides, bases, acids, and salts).

The term “processing” refers to the passage of the analyte through a fluid flow pathway, where the analyte is exposed to various reagents that interact with the analyte, e.g., denaturing reagents, neutralizing reagents, fragmentation reagents, lysing reagents, deglycosylation reagents, purification reagents, etc. The processed analyte is then delivered to the lateral flow strip via the sample delivery array.

As used herein, the term “processed analyte” refers to an analyte that has been processed by a fluid flow pathway, and can be detected by the pressure driven fluidics device.

As used herein, the term “fluid” refers to a biological solution derived from a biological source comprising one or more analytes. Biological sources include materials derived from biological organisms, including but not limited to blood samples, urine, saliva, or other glandular secretions and products, semen, bronchoalveolar fluid, cerebrospinal fluid, medullar fluids, mucosal area secretions, and feces. Biological sources can also be derived from an environmental source (e.g. water, dirt, etc.)

In the present invention, “fluid” is defined as a liquid. In one example, the fluid is water, with or without the addition of other components. These additional components may include, but are not limited to, nutrients, salts, surfactants, and protease inhibitors, needed to support stability and assist with processing of the biological sample. In other embodiments, a fluid is a buffer mixed with a biological sample, such as, for example, a blood sample.

As used herein, the term “fluid flow pathway” refers to one or more chambers in fluid communication with each other (e.g., the chambers are connected by flow regulation tubes, filters, or a combination thereof), and in which processing of the analyte occurs. As the analyte passes through each chamber, it undergoes processing, e.g., denaturation, fragmentation, deglycosylation, neutralization, purification, etc. In some examples, a fluid flow pathway is connected to one or more channels of the lateral flow component via a sample delivery array.

As used herein, the term “denaturation chamber” refers to one or more regions of the device where an analyte or a solution comprising the analyte may be processed, e.g., by a reaction to modify its native conformation. As used herein, the term “denaturing reagent” refers to reagents that are capable of interacting with the analyte and changing its structural conformation (including but not limited to, guanidine-based denaturing solutions, formamide-based solutions, detergent-based solutions, urea-based solutions, disulfide bond reducers, agitation, radiation, or acid, bases, solvents, cross-linking reagents, chaotropic reagents, disulfide bond reducers, mechanical agitation, radiation, temperature. As used herein, the term “denature” refers to a change in structure as compared to the original/native/unprocessed conformation of the analyte.

As used herein, the term “neutralization chamber” refers to one or more regions of the device wherein an analyte or a solution comprising the analyte may be processed, by a reaction to neutralize or negate the activity of one or more of the “denaturing reagents.” As used herein, the term “neutralization reagent” refers to reagents that are capable of interacting with the analyte and/or “denaturing reagents” and neutralize or negate its activity (acid, bases, solvents, cross-linking reagents, buffering agents). For example, the term “neutralize” refers to the action of correcting the pH to neutral values, or otherwise negate the activity of one or more “denaturing reagents.”

As used herein, the term “purification chamber” refers to one or more regions of the device reserved to separate one or more components. As used herein, the term “purification reagent” refers to reagents that interact with the solution containing the biological sample containing the analyte to allow contaminants to be separated using components including, but not limited to: size or charge filters, resins, binding agents such as lectins and proteins, antigens, and antibodies. As used herein, the term “purify” means to separate from the remaining components.

As used herein, the term “fragmentation chamber” refers to one or more regions of the device where the analyte is broken or cleaved to smaller pieces. As used herein, the term “fragmentation reagent” or “cleavage reagent” refers to reagents that are capable of breaking the analyte into smaller parts including, but not limited to: enzymes, mechanical agitation, temperature, acids, or base-based treatments. As used herein, the term “fragment” refers to breaking the analyte into smaller pieces.

As used herein, the term “impurity removal chamber” refers to one or more regions of the device where impurities may be completely or partially removed from the solution comprising the analyte. As used herein, the term “impurity removal reagent” refers to a mechanism or agent that removes impurities including, but not limited to: filters, resins, binding agents such as lectins and proteins, and antibodies, etc. As used herein, the term “impurity” refers to anything other than the analyte to be detected in a given detection channel, and in some embodiments, a substance that can cause a false positive.

As used herein, the term “solubilization chamber” refers to one or more regions of the device where the analyte is solubilized into the fluid. As used herein, the term “solubilization reagent” refers to a surfactant that acts to reduce the surface tension of water, and in this case, specifically includes emulsifiers or dispersants for oils or solids, respectively (e.g., anionic surfactants including, but not limited to, soaps, carboxylates, sulfonaiion, sulfatation, sulfates, sulfonates, etc.; nonionic surfactants including, but not limited to, ethoxylated alcohols, alkylphenols, fatty acid esters, etc.; cationic surfactants including, but not limited to, linear alkyl-amines, alkyl-ammoniums, etc.). As used herein, the term “solubilize” means to make soluble in solution.

As used herein, the tern “deglycosylation chamber” refers to one or more regions of the device where the analyte undergoes full or partial deglycosylation. As used herein, the term “deglycosylation reagent” refers to a chemical or enzyme that can remove glycans from a glycoprotein (e.g., PNGase F, O-Glycosidase, Neuraminidase, Galactosidase, β-N-acetylglucosaminidase, trifluoromethanesulphonic acid, etc.). As used herein, the term “deglycosylation” refers to partial or total removal of glycan from protein.

As used herein, the term “lysis chamber” refers to one or more regions of the device where cells comprising the analyte are ruptured to release the components of the cell into the fluid. As used herein, the term “lysis reagent” refers to chemical(s) capable of lysing a cell membrane and releasing contents into solution (e.g., NP-40, Triton X-100, digitonin, saponin, CHAPS, SDS, sodium deoxycholate, etc.). As used herein, the term “lyse” refers to dissolution or disruption of cell membranes.

As used herein, the term “precipitation chamber” refers to one or more regions of the device where analytes or non-analytes are removed from solution, or deposited in solid form from solution. As used herein, the term “precipitation reagent” refers to chemical(s) that induce precipitation of analytes or non-analytes from solution (e.g., polyvinylpolypyrrolidone, ammonium sulfate, trichloroacetic acid, etc.). As used herein, the term “precipitate” refers to an insoluble solid that emerges from a solution.

As used herein, the term “sample pre-treatment zone” refers to a region of a lateral flow strip where the biological sample comprising the processed analyte can be subjected to interaction with antibodies, or other binding molecules, that can remove unwanted or competitor molecules from the fluid, e.g., prior to coming into contact with the analyte readout bands.

As used herein, the term “pressure” refers to application of a physical force to cause movement (or dislocation) of a fluid comprising an analyte, biological source and/or other reagents through the pressure driven component of the device. As used herein, “manually applied pressure” does not rely on an external electrical power source but instead is generated by a non-electrical power based application of pressure to drive the flow of fluid through one or more fluid flow pathways, e.g., such as by applying pressure to a syringe that connects to the pressure driven fluidics device.

As used herein, “controlled pressure” is defined as pressure applied to a fluid moving through the pressure driven component of the device such that the pressure drop along the fluid flow pathway is held constant. Thus, as resistance to flow in the pathway is increased, rather than continuing to apply increasing pressure to keep the flow rate constant, the flow rate is reduced such that the pressure remains constant. As used herein, a constant pressure includes pressure that varies. For example, the pressure may “pulse” at a given frequency, for example, but the average pressure will remain constant.

As used herein, the term “capillary action” or “wicking action” or “lateral flow” refers to movement of fluid in response to capillary force without applied force such as pressure. For example, in some embodiments, the fluid comprising the analyte flows down the length of the lateral flow strip for purification (optional) and detection.

As used herein, the term “channel” refers to one or more regions of the device where processed analytes are detected. Typical dimensions for a channel include but are not limited to 5 mm-7 mm in width and by 70 mm-100 mm in length. The channel is able to be viewed through the top casing of the device.

As used herein, the term “chamber” refers to one or more delineated regions of the device where analytes will be processed, as described herein. Typical dimensions for a chamber include but are not limited to a cylinder 3 mm in diameter and 5 mm high. Chambers also have input and output ports to connect to flow regulation tubes. in some embodiments, the chambers comprise an input port at or near the bottom of a chamber and an output port at or near the top of the chamber.

As used herein, the term “collection tube” refers to a tube used to collect and temporarily hold the fluid/biological solution to be run on the pressure driven fluidics device. In sonic embodiments, the collection tube may be the barrel of the syringe.

As used herein, the term “readout bands” or “detection bands” refer to regions of the device where the presence of the desired analyte will be revealed through an accumulation of the analyte together with labeled detection antibodies or molecules. The detection label will accumulate at the “readout band” site creating a detectable band through a change in color or emission of fluorescence. A positive “readout band” will appear if the reaction occurs in the presence of the analyte while no “readout band” will be detected if the analyte is not present or if the analyte is present at undetectable levels.

As used herein, the term “affixed” refers to the process of binding a capture molecule (antibody or other molecule that has affinity for a given analyte) to a surface (e.g., a chamber, a channel, a lateral flow strip, etc.) using covalent or non-covalent linkage for purification or for creation of a readout band to detect a given analyte.

As used herein, the term “flow regulation tubes” refer to tubes that permit the flow of liquid from one chamber to another within a given fluid flow pathway and thereby control the rate of flow and reaction time. As used herein, flow regulation tubes do not contain valves.

In the present invention, “flow” is defined as movement of the fluid through the device, whether by applied pressure or capillary action. In the present invention, “flow rate” is defined as the volume of a fluid moving along a surface per unit time.

As used herein, the term “in fluid communication with” refers to two spatial regions being connected together such that a liquid may flow between the two spatial regions. For example, a channel may be in fluid communication with a chamber such that a fluid may freely flow into the fluidic channel from the reaction chamber. The term “in fluid communication with” also includes two spatial regions being in fluid communication through one or more filters, resins, flow regulation tubes, or other fluidic components that are configured to control or regulate a flow of fluid through a system. In some cases, two spatial regions may be in fluid communication with each other even if, under certain conditions, a certain fluid would not be able to flow freely into a spatial region.

As used herein, the term “active infection” refers to infections associated with current clinical symptoms and/or presence of the pathogen. Examples of active infections include, but are not limited to acute or recent infections by viruses, parasites, fungi and bacteria.

As used herein, the term “past infection” refers to non-active infections, and whose clinical presentation does not support an active infection as determined by laboratorial exams and/or clinical symptoms. Examples of past infections include, but are not limited to previous infection by viruses, parasites, fungi and bacteria.

As used herein, the term “cancer specific antigen” refers to antigens/molecules that are uniquely expressed or more abundantly expressed by cancerous cells or by non-cancerous cells, generally, under the influence of a cancer-associated environment. Examples of cancer specific antigens include, but are not limited to viral oncogenes, melanin, fetal oncogenes (e.g., alphafetoprotein, carcinoembryonic antigen, etc.), proto-oncogenes, cancer-testis antigens (CTAG1B), and mutant genes (altered ras, p53). A list of cancer-specific antigens is provided below.

As used herein, the term “non-communicable disease” refers to diseases that cannot be transmitted from one person to another or are non-infectious. Examples of non-communicable diseases include, but are not limited to any non-pathogen associated diseases such as genetic diseases, cardiovascular disease, hypertension, diabetes, cancers, obesity, and chronic kidney disease.

As used herein, the terra “pathogen” refers to organisms that can cause a disease. Examples of pathogens include, but are not limited to bacteria, virus, prions, parasites and fungi.

As used herein, the term “viscosity” refers to the consistency of a fluid and its resistance to flow.

As used herein, the term “detergent” refers to any group of organic or inorganic-based substances that have emulsifying properties. Examples of detergents include, but are not limited to anionic, cationic or non-ionic based detergents. e.g., SDS, Triton X-100, CTAB, DTA, etc. Detergents may act to denature the analyte.

As used herein, the term “acidify” refers to bringing or lowering the pH to some value between 0 and 7 of a reference neutral liquid.

As used herein, the terra “basify” refers to bringing the raising the pH to some value above 7 of a reference neutral liquid.

As used herein, the term “conjugate pads” refer to a pad designed to permit the interaction between the analyte and a detector reagent before moving into the lateral flow strip. In some embodiments, the conjugate pad will be made of compressed fibers of glass or cellulose.

As used herein, the term “sample delivery array” refers to a set of tubes designed to deliver the fluid comprising the processed analyte into the conjugate pads for each channel.

As used herein, the term “test cluster” refers to a group of channels that receive a given fluid sample after processing by a common fluid flow pathway, e.g., the first fluid flow pathway or the second fluid flow pathway.

As used herein, the term “lateral flow strip” refers to a strip of material used to fit within a channel for detection of a given analyte. Lateral flow strips may be made from a variety of materials, including but not limited to, nitrocellulose and modified nitrocellulose such as plastic-backed nitrocellulose, polyvinylidene fluoride, nylon, and polyethersulfone.

As used herein, the term “wick” refers to an absorbent pad that will increase the volume of sample beyond the volume of the lateral flow strip and will be made from cellulose filters.

As used herein, the term “readout window” refers to the location on the device where a result (positive or negative) can be observed.

As used herein, the term “housing” refers to the support structure for holding the components of the pressure driven fluidics device including the fluid flow pathways and channels. In some embodiments, the housing is composed of a plastic housing and adhesive board for supporting the components. In other embodiments, one or more components are integrated with the plastic housing.

As used herein, the term “detection” refers to the action or process of revealing the presence of a given analyte, e.g., via a colorimetric assay,

As used herein, the term_{—} “pre-treatment” refers to treatments to which the fluid can be submitted to before the detection step.

As used herein, the term “IgG” refers to a class of antibodies that bear the heavy chain gamma.

As used herein, the tem) “IgM” refers to a class of antibodies that bear the heavy chain mu.

As used herein, the term “IgE” refers to a class of antibodies that bear the heavy chain epsilon.

In the present invention, “deposit” includes a deposit of a reagent on an inside surface of a chamber that comes into contact with the fluid containing analyte. Deposited reagents may include resins, binding agents such as lectins and proteins, and antibodies, as well as various reagents used for processing in the first or second fluid flow pathway.

In the present invention, “detection compound” is defined as any compound added to the test system for detecting the presence of the analyte. These detection compounds may be pharmaceutical compounds, small molecules, or biological compounds. Some examples include peptides, proteins, peptidomimetics, antibodies, non-antibody specific binding molecules, such as adnectins, affibodies, avimers, anticalins, tetranectins, DARPins, mTCRs, engineered Kunitz-type inhibitors, nucleic acid aptamers and spiegelmers, peptide aptamers and cyclic and bicyclic peptides and small synthetic or natural organic molecules (Ruigrok et al. Biochem J. (2011) 436, 1-13; Gebauer et al., Curr Opin Chem Biol. (2009) (3):245-55.)

In the present invention, “antibody” refers to immunoglobulin molecules and immunologically active portions of immunoglobulin molecules, i.e., molecules that contain an antigen binding site that immunospecifically bind to an antigen. As such, the term antibody encompasses not only whole antibody molecules, but also antibody fragments as well as variants (including derivatives) of antibodies and antibody fragments. Examples of molecules which are described by the term “antibody” in this application include, but are not limited to: single chain Fvs, and fragments comprising or alternatively consisting of, either a VL or a VH domain. The term “single chain Fv” or “scFv” as used herein refers to a polypeptide comprising a VL domain of an antibody linked to a domain of an antibody. Antibodies of the invention include, but are not limited to, monoclonal, multispecific, human or chimeric antibodies or antibodies made in animals, single chain antibodies, Fab fragments, F(ab′) fragments, antiidiotypic (anti-Id) antibodies (including, e.g., anti-Id antibodies to antibodies of the invention), and epitope-binding fragments of any of the above. The immunoglobulin molecules of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule.

As used herein, the term “antibody” or “antigen binding domain” refers to immunoglobulin molecules and immunologically active portions of immunoglobulin molecules, i.e., molecules that contain an antigen binding site that immunospecifically bind an antigen. As such, the term antibody encompasses not only whole antibody molecules, but also antibody fragments as well as variants (including derivatives) of antibodies and antibody fragments. Examples of molecules which are described by the term “antibody” in this application include, but are not limited to: single chain Fvs (scFvs), Fab fragments, Fab′ fragments, F(ab′)2, disulfide linked Fvs (sdFvs), Fvs, and fragments comprising or alternatively consisting of, either a VL or a VH domain. The term “single chain Fv” or “scFv” as used herein refers to a polypeptide comprising a VL domain of antibody linked to a VH domain of an antibody.

Antibodies of the invention include, but are not limited to, monoclonal, multispecific, bi-specific, human, humanized, mouse, rabbit, chicken, phage display generated, or chimeric antibodies, single chain antibodies, Fab fragments, F(ab′) fragments, antiidiotypic (anti-Id) antibodies (including, e.g., anti-Id antibodies to antibodies of the invention), and epitope-binding fragments of any of the above. The immunoglobulin molecules of the invention can be of any type (e.g., IgG, IgM, IgD, IgD, IgA and IgY), class (e.g., IgG1, IgG2, IgG3, IgG4, IgA1 and IgA2) or subclass of immunoglobulin molecule.

As used herein, the terms “a” or “an” are used, as is common in patent documents, to include one or more than one, independent of any other instances or usages of “at least one” or “one or more.”

As used herein, the term “or” is used to refer to a nonexclusive or, such that “A or B” includes “A but not B,” “B but not A,” and “A and B,” unless otherwise indicated.

As used herein, the term “about” is used to refer to an amount that is approximately, nearly, almost, or in the vicinity of being equal to or is equal to a stated amount, e.g., the state amount plus/minus about 5%, about 4%, about 3%, about 2% or about 1%.

### B. Device

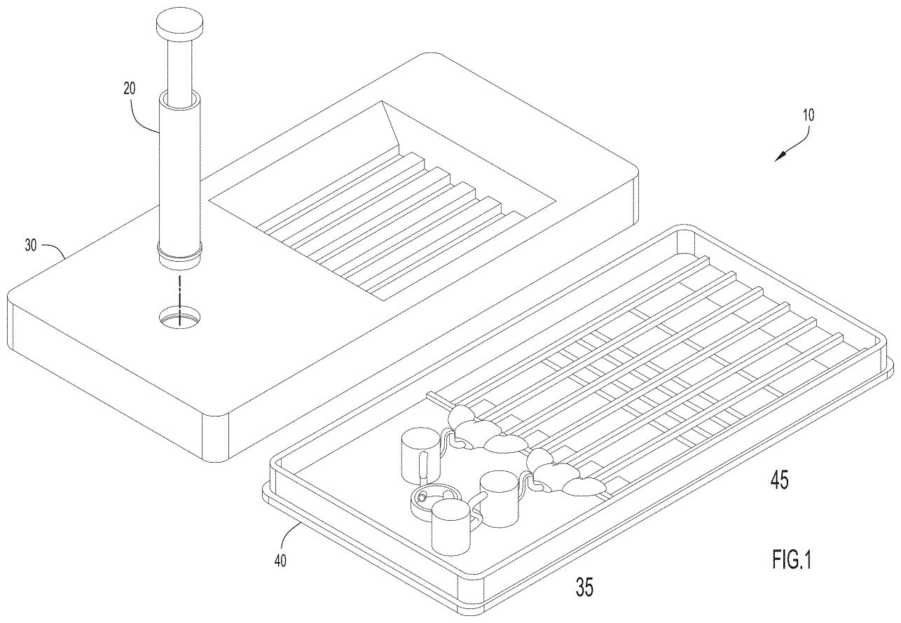

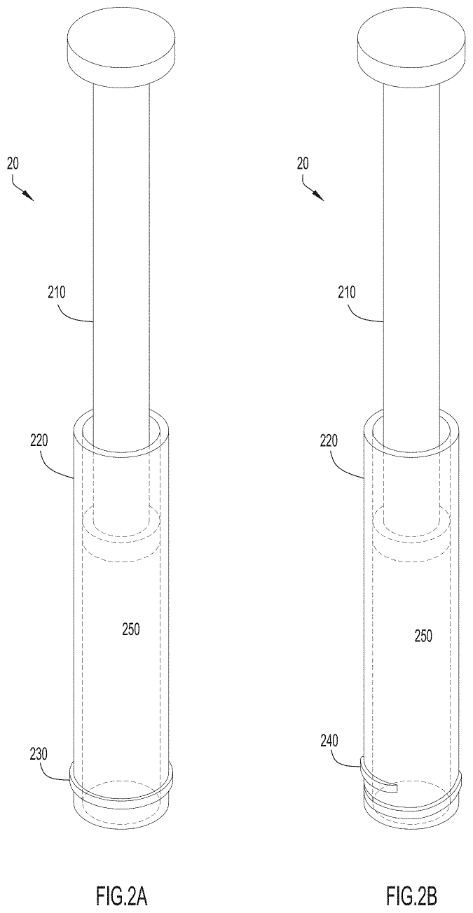

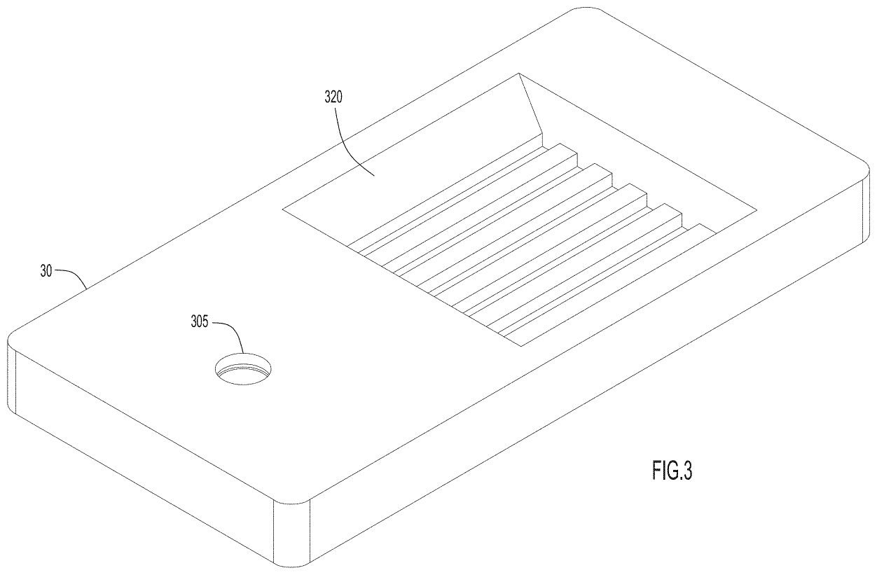

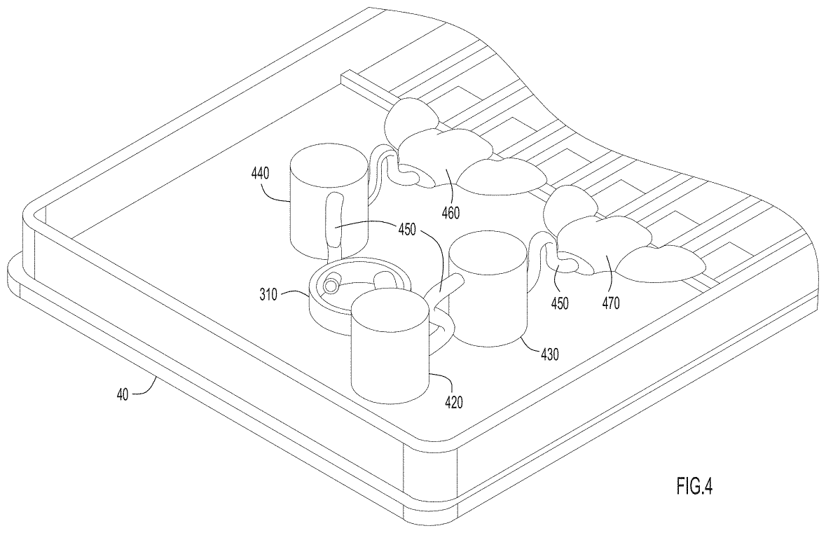

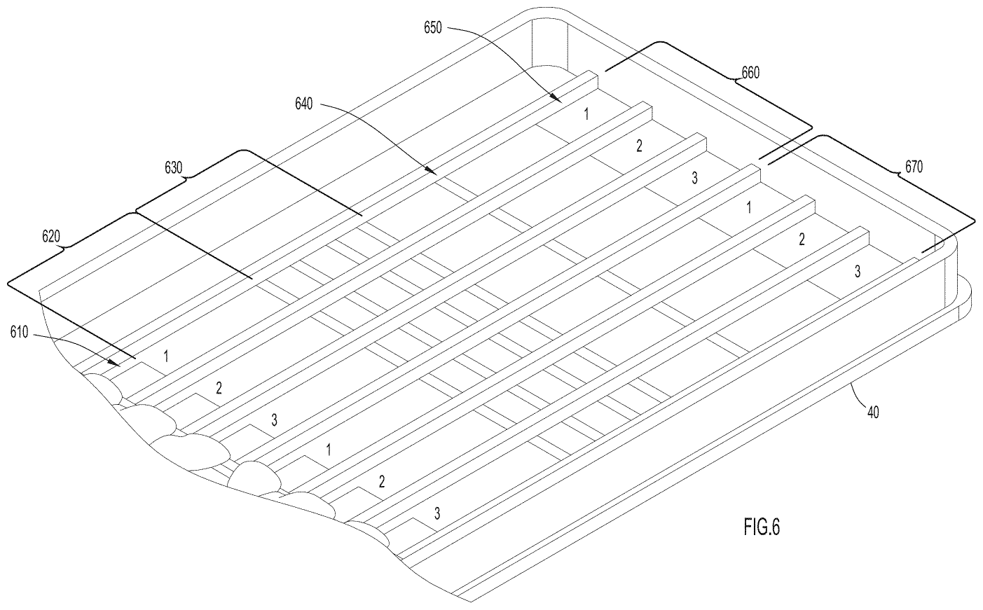

FIG. 1 is an illustration of an exemplary pressure driven fluidics device 10, according to embodiments of the invention. A syringe 20, containing the analyte in a buffered solution or fluid, attaches to the top casing 30 of the pressure driven fluidics device at input port 305 (also shown in FIG. 3), which connects to an input chamber 310 (see, e.g., FIG. 4). The top casing attaches to the bottom casing 40 of the pressure driven fluidics device. The components of the pressure driven fluidics device 10 are described herein in reference to FIGS. 2-7. The portion of the device labeled as portion 35 includes pressure driven components. Fluid flows through this portion of the device based on applied pressure. The portion of the device labeled as portion 45 includes lateral-flow driven components. Fluid flows through this portion of the device based on capillary action.

In some embodiments, input port 305 is configured to connect to a syringe. In still other embodiments, input port 305 is configured to connect to a pipette or other collection tube. In yet other embodiments, input port 305 is configured to connect to tubing having a UNF thread which is connected to a syringe. The syringe applies pressure to cause the analyte-containing solution to flow into the device. In general, present invention embodiments include any non-electrical pump capable of applying pressure to the analyte-containing fluid to cause the fluid to flow into input port 305.

FIGS. 2A-2B are illustrations of two example different syringe types that may connect to input port 305. FIG. 2A is an illustration of an example syringe 20 comprising a plunger 210, a barrel 220 and a “snap-in” tip 230. The snap in tip 230 connects to input port 305. FIG. 2B is an illustration of an example syringe 20 comprising a plunger 210, a barrel 220 and a luer-lock type tip 240. The luer-lock tip 240 is configured to screw into input port 305.

The plunger 210 may be extended such that fluid 250 is drawn into the interior of the syringe. Once the syringe is connected to the input port 305, manual pressure may be applied, depressing the syringe, and driving fluid flow through the interior of the device. Lines or notches on the barrel may indicate how far the syringe is to be depressed at each stage to control the flow rate of fluid into the device. Thus, in some embodiments, depression of the plunger 210 serves as the energy source for the microfluidics pathway of portion 35 of FIG. 1.

In some embodiments, the fluid comprising the analyte is mixed with a liquid buffer (running buffer) prior to being introduced into the device, to increase the overall volume of fluid and prevent coagulation of blood (e.g., a ACD, or EDTA buffer).

FIG. 3 is an illustration of top casing 30 shown in additional detail. Top casing 30, which attaches to bottom casing 40, comprises input 305 port for connecting to a syringe or other source for applying pressure to a fluid to cause the fluid to flow through the interior of the device. Top casing 30 may be formed from any suitable material. In preferred embodiments, the material is plastic.

Top casing 30 also comprises a readout window 320. which allows identification of readout bands and control bands. Readout window 320 may be formed from any suitable material that is transparent. In preferred embodiments, the material is clear plastic. In some embodiments, readout window 320 may be an uncovered opening in the top case of device 10. In general, readout window 320 is of a sufficient size to allow the readout bands and the control bands to be visualized.

FIG. 4 is an illustration of bottom casing 40 shown in additional detail. Bottom casing 40, to which top casing 30 is attached to, comprises a plurality of chambers 310, 420, 430, and 440, flow regulation tubes 450, and sample delivery arrays 460 and 470. In this example, two distinct processing pathways are shown. The first processing pathway comprises chambers 420 and 430, while the second flow regulation pathway comprises chamber 440. In some embodiments, the first processing pathway may comprise 2, 3, 4, 5, 6, 7, 8, 9, and 10 chambers, each chamber connected by a flow regulation tube 450. Tubes may be formed by a 3-D printer, by micro molding, or by inserting actual tubing. In some embodiments, the second processing pathway may comprise 2, 3, 4, 5, 6, 7, 8, 9, and 10 chambers, each chamber connected by a flow regulation tube 450. In general, the first processing pathway will be different from the second processing pathway.

Bottom casing 40 holds the processing chambers, flow regulation tubes, sample delivery array, and lateral flow strips. in some embodiments, the chambers are attached to the casing, via an adhesive. In other embodiments, the chambers are formed in an integrated manner with the device. Each component is described in additional detail herein. Bottom case 40 may be formed from any suitable material. In preferred embodiments, the material is plastic.

In the example embodiment shown in FIG. 4, chamber 310 is a sample/or analyte recipient well. This chamber receives fluid from the syringe via input port 305. In some embodiments, chamber 310 may hold a volume up to 200 ul of buffer/analyte solution 250. In other embodiments, chamber 310 may hold a volume ranging from 10 ul to 1 ml, preferably from 50 ul to 500 ul, and more preferably from 100 ul to 300 ul.

In some embodiments, chamber 310 contains a pre-filter for removal of unwanted particles (debris) or cells (RBC, WBC, etc.) or the like. The pre-filter may sit at the top of the chamber 310. Alternatively, the pre-filter may be present at the entrance to the flow regulation tubes 450.

For example, a filter, e.g., a glass fiber filter, or other type of filter may be placed into device prior to processing, to remove particles or cells that should not be passed into the fluid flow pathway. For whole blood, this filter would remove RBCs, for example, from the whole blood, allowing plasma to pass into the system. In other cases, white blood cells would be removed or permitted to pass into the system, depending on the analysis.

Chamber 310 connects to two chambers in two distinct processing pathways, e.g., the first fluid flow pathway and the second fluid flow pathway. In this example, chamber 310 connects to chamber 420 via flow regulation tubing 450, and chamber 420 connects to chamber 430 via flow regulation tubing 450. Thus, in this embodiment, the first processing pathway comprises chambers 420 and 430. Chamber 430 connects to sample delivery array 470, which delivers processed samples (by chambers 420 and 430) to conjugate pads. It is expressly noted that the first fluid flow pathway is not limited to two chambers, additional chambers comprising different processing reagents may be added to the first fluid flow pathway by connecting the additional chambers with flow regulation tubing as described herein. Further, in other embodiments, the first fluid flow pathway may comprise a single chamber.

Chamber 310 also connects to chamber 440 via flow regulation tubing 450. In this example, the second processing pathway comprises a single chamber 440. Chamber 440 connects to sample delivery array 460, which delivers processed samples to conjugate pads. It is expressly noted that the second fluid flow pathway is not limited to two chambers; additional chambers comprising different processing reagents may be added to the second fluid flow pathway by connecting the additional chambers with flow regulation tubes.

In general, the fluid comprising one or more analytes is pumped through the first processing pathway and the second processing pathway, wherein the same analyte, or different analytes, are processed in two different pathways using different processing steps and reagents. Thus, in some cases, the same analyte will be processed in two different fluid flow processing pathways. However, in other embodiments, one analyte will be processed in a first fluid flow processing pathway and a different analyte will be processed in a second fluid flow processing pathway. For example, for detecting Zika antigens, the first fluid flow pathway will treat the Zika NS1 antigen with specific anti-NS1 antibodies against deglycosylated or fragmented NS1 in order to detect the antigen(s), and the second fluid flow pathway will contain reagents to remove anti-Dengue cross reactive antibodies (e.g., using a ligand specific for the anti-Dengue antibodies) for subsequent detection of anti-NS1 Zika antibodies.

Flow regulation tubing 450 is used to connect the chambers to each other, as well as the chambers to the sample delivery arrays 460 and 470. In some embodiments, flow regulation tubes may connect from the bottom of a chamber to the top of a subsequent chamber to minimize formation of trapped air bubbles within the tubing. Thus, flow regulation tubes control volume and flow rate of liquids between chambers and delivery arrays.

Other processing steps, including dilution of the analyte may be included in the first processing pathway with the syringe or an optional dilution chamber. The first and second processing pathways process the same sample in different ways in order to conduct different detection tests on the analyte(s) (e.g., the first fluid flow processing pathway may produce a fragmented analyte for detection, and the second fluid flow pathway may produce an analyte retaining its non-fragmented form for detection, or prepare a distinct analyte for detection) during the lateral flow detection process.

In one embodiment, chamber 420 may contain reagents suitable for a denaturing process, and. chamber 430 may contain reagents suitable for a neutralization process. Chamber 440 may comprise reagents for impurity removal. Thus, the sample may be processed according to two different processes, and subsequently detected by a lateral flow strip comprising test bands suitable to test for each type of processed analyte. Additional examples of flow processing paths are discussed below.

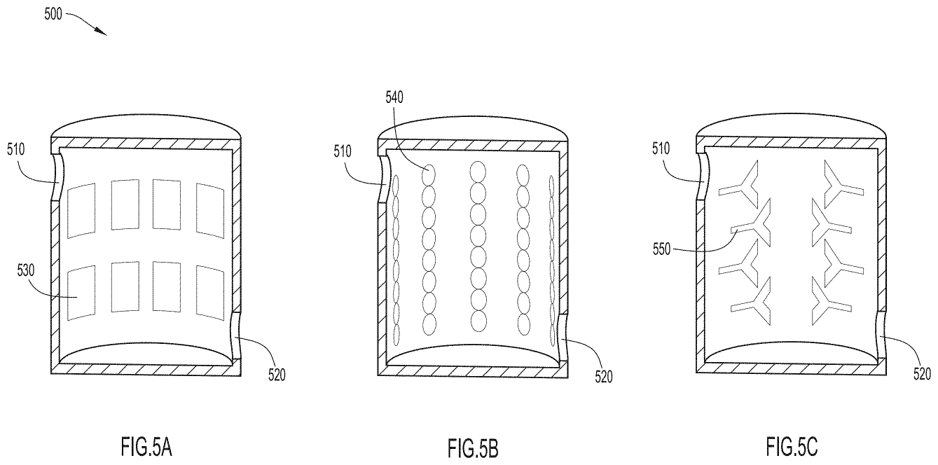

FIGS. 5A-C are illustrations of various embodiments of chambers 500. According to embodiments of the present invention, chambers comprise one or more reagents for processing steps selected from the group consisting of: denaturing, neutralization, impurity removal, solubilization, acidification or basification, precipitation, fragmentation, dilution, lysis, etc. A fluid processing flow comprises any one or more of the processing steps with any one or more reagents in any one or more channels.

In some embodiments, each chamber is configured for a single processing step (e.g., a chamber for denaturing, a chamber for neutralization, a chamber for impurity removal, a chamber for solubilization, a chamber for precipitation, a chamber for fragmentation, a chamber for dilution, etc.) in other embodiments, multiple processing steps may occur in a single chamber, e.g., lysis and denaturing.

Chambers may be coated with reagents needed for a particular processing step. The reagent may be in a liquid form (e.g., a reagent in a membrane that ruptures under a fluid-flow pressure induced by the syringe), in dried form (e.g., as the fluid comprising the analyte is propelled through the fluid flow pathway by pressure from a syringe or pump, where the fluid dissolves reagents deposited and dried on the interior surface of the chamber) or immobilized (e.g., microbeads, small molecules, antibodies bound to the interior surface of the chamber, etc.). Thus, in general, reagents may be supplied in liquid, dried, or immobilized form.

FIG. 5A shows an illustration of an example chamber 500 with an input port 520 to connect to flow regulation tubes and an output port 510 to connect to flow regulation tubes, and deposits of dry-reagents 530 on the side of the chamber.

FIG. 5B shows an illustration of an example chamber 500 with an input port 520 to connect to flow regulation tubes and an output port 510 to connect to flow regulation tubes, and microbeads or other resin 540 on the side of the chamber.

FIG. 5C shows an illustration of an example chamber 500 with an input port 520 to connect to flow regulation tubes and an output port 510 to connect to flow regulation tubes, and antibodies 550 (not to scale) on the side of the chamber.

Reagents may include catalysts, inhibitors, surface coatings (e.g., Poly(ethylene glycol), poly(vinylpyrrolidone), Polyvinylamine, 76 epoxysilane 38, Fluoropolymer and carbon black, etc.), antibodies, labelled-antibodies, proteins (e.g., avidin, protein A, protein G, BSA, synthetic peptides, antigens, laminin, fibrinogen, poly-L-lysine), enzymes, DNA, buffers (PBS, Tris-Hcl/Tris-EDTA, lysis buffer, elution buffer, wash buffer), chemical and particles, buffers, chemical additives, labelling agents, gels and particles, reagents to adjust pH, organic solvents, photoreactive agents (in which case the chamber would be protected from exposure to light), salts (e.g., NaCl, KCl, MgCl2, CaCl2, Sucrose, Trehalose, Dextran, etc.), surfactants, gels, stabilizers, surfactants (e.g., Tween 20, Triton-X, Pluronic, etc.), antioxidants, particles (e.g., gold nanoparficles with antibodies or aptamers, antibody-conjugated particles, etc.).

In some embodiments, volumes of liquid reagents that are incorporated into microfluidic devices range from picoliters to several hundred microliters. In some embodiments, volumes of solid reagents that are incorporated into microfluidic devices range from picograms to micrograms. Once formed, the chambers may be coated with various reagents for processing as described herein.

FIG. 6. is an illustration of the lateral flow component of bottom casing 40. In this figure, conjugate pads 610 (three lanes, labeled 1, 2, 3, for the first fluid flow processing pathway (shown by test cluster 670); and three lanes, labeled 1, 2, 3, for the second fluid flow processing pathway (shown by test cluster 660)) are attached to the lateral flow strip, and receive liquid sample from sample delivery array 460 or 470. In some embodiments, conjugate pads 610 deliver specific detection conjugated molecules to the lateral flow strip. In some embodiments, the conjugate pad delivers antibodies conjugated with colloidal gold to the lateral flow assay. As the fluid containing analyte comes into contact with the conjugate pad, the antibody conjugated with colloidal gold, the detecting antibody, binds to the analyte/antigen. It is understood that the antibodies conjugated with colloidal gold are readily released from the conjugate pad, allowing the analyte-antibody complex to migrate along the lateral flow strip, e.g., a nitrocellulose membrane, towards test cluster 660 or test cluster 670. In some embodiments, the lateral flow strip is a nitrocellulose strip including components 610, 620, 630, 640 and 650.

In some embodiments, the analyte-antibody complex migrates from the conjugate pad 610 through a pretreatment region 620. The pre-treatment region may include reagents (e.g., antibodies, ligands, etc.) to remove additional impurities from the fluid that could cause false positive or false negative results.

Once passing through pretreatment region 620, the fluid comprising the analyte flows along the lateral flow strip until coming into contact with analyte positive result readout bands 630. In sonic embodiments, the readout bands 630 may comprise a single type of capturing antibody, or multiple types of capturing antibodies (e.g., one antibody type per readout hand or multiple types of antibodies per readout band), which act to increase the local concentration of the same analyte-antibody complex. In other embodiments, multiple readout bands may be present, each readout band including a different type of capturing antibody (or multiple types of capturing antibodies) with each capturing antibody directed toward a different analyte. In still other embodiments, single or multiple readout bands may be present, each readout band composed of a distinct type of antigen or multiple types of antigens to capture antibodies (e.g., generated by a patient) directed against the antigen(s). In sonic embodiments, the readout band becomes visible as part of a colorimetric assay, e.g., the readout band may turn red, purple, etc. once coming into contact with the analyte-antibody complex.

In sonic embodiments, the excess detection antibody or other positive control not captured by the readout bands continues to migrate down the nitrocellulose membrane, coming into contact with positive control readout band 640. The positive control readout band indicates that the reagents, sample processing and lateral flow are all functioning according to standard processes. Residual solution continues to migrate down the lateral flow strip to wick 650. Once reaching the end of the lateral flow strip, fluid may exit the device through a port in casing 40 (not shown).

In this example, test cluster 660 corresponds to analytes processed by chambers 310 and 440. Readouts for 3 distinct sets of analyte detection are shown. Test cluster 670 corresponds to analytes processed by chambers 310, 420 and 430. Readouts for 3 distinct sets of analyte detection are shown.

### C. Processing Pathways

According to embodiments of the invention, the device comprises at least a first fluid flow pathway and a second fluid flow processing pathway. Examples of various processing configurations for the first fluid flow pathway are provided as follows. In general, the first fluid flow pathway may perform one or more of the following processing steps: neutralization, denaturation, adjustment, lysis, precipitation, solubilization, fragmentation, etc. In this section (B), tubing may refer to tubes integrated with the device or tubing that is formed separately and connects the components of the device.

According to an example embodiment, the analyte (mixed with the fluid) flows from the input chamber through flow regulation tubing into a denaturation/lysis chamber. In this chamber, denaturation and lysis reagents that have been deposited on the walls of the chamber mix with the fluid to lyse the cells, releasing the analyte into the fluid. Once released from the cell, the analyte is denatured. The denatured analyte then exits the first chamber by flowing through flow regulation tubing into a second chamber, where it undergoes neutralization by mixing with neutralization reagents that have been deposited into the second chamber. Once the analyte has been neutralized, the analyte flows via flow regulation tubing into the sample delivery array for distribution into the lateral flow assay. In this example, the lateral flow assay comprises a reagent to detect, for example, a linear epitope of a denatured analyte.

According to another example embodiment, the analyte (mixed with the fluid) flows from the input chamber through flow regulation tubing into a denaturation chamber. In this chamber, denaturation reagents that have been deposited on the walls of the chamber mix with the fluid to denature the analyte. In this example, the analyte may be a secreted extracellular protein, a protein on the surface of a cell, or a protein attached to a bead, or other protein in solution. The denatured analyte then exits the first chamber by flowing through flow regulation tubing into a second chamber, where it undergoes neutralization by mixing with neutralization reagents that have been deposited into the second chamber. Once the analyte/denaturation reagents have been neutralized, the analyte flows via flow regulation tubing into the sample delivery array for distribution into the lateral flow assay. In this example, the lateral flow assay comprises a reagent to detect, for example, a linear epitope of a denatured analyte.

According to another example embodiment, the analyte (mixed with the fluid) flows from the input chamber through flow regulation tubing into a lysis chamber. In this chamber, lysis reagents that have been deposited on the walls of the chamber mix with the fluid to lyse cells comprising the analyte. The analyte then exits the first chamber by flowing through flow regulation tubing into a second chamber, where it undergoes fragmentation by mixing with fragmentation reagents that have been deposited into the second chamber. Once the analyte has been fragmented, the analyte flows via flow regulation tubing into the sample delivery array for distribution into the lateral flow assay. In this example, the lateral flow assay comprises a reagent to detect, for example, a three-dimensional epitope or configuration of the analyte.

According to another example embodiment, the analyte (mixed with the fluid) flows from the input chamber through flow regulation tubing into a first chamber, a lysis chamber. In this chamber, lysis reagents that have been deposited on the walls of the chamber mix with the fluid to lyse the cells, releasing the analyte into the fluid. The analyte then exits the first chamber by flowing through flow regulation tubing into a second chamber, where it undergoes denaturation by mixing with denaturization reagents that have been deposited into the second chamber. The denatured analyte then exits the second chamber by flowing through flow regulation tubing into a third chamber, where it undergoes neutralization by mixing with neutralization reagents that have been deposited into the third chamber. Once the analyte has been neutralized, the analyte flows via flow regulation tubing into the sample delivery array for distribution into the lateral flow assay. In this example, the lateral flow assay comprises a reagent to detect, for example, a linear epitope of a denatured analyte.

According to another example embodiment, the analyte (mixed with the fluid) flows from the input chamber through flow regulation tubing into a first chamber, where it undergoes fragmentation by mixing with fragmentation reagents that have been deposited into the first chamber. Once the analyte has been fragmented, the analyte flows via flow regulation tubing into the sample delivery array for distribution into the lateral flow assay. In this example, the lateral flow assay comprises reagents to detect, for example, two or more three dimensional or linear epitopes of an analyte.

According to another example embodiment, the analyte (mixed with the fluid) flows from the input chamber through flow regulation tubing into a first chamber, where it undergoes solubilization by mixing with solubilization reagents that have been deposited into the first chamber. Once the analyte has been solubilized, the analyte flows via flow regulation tubing into the sample delivery array for distribution into the lateral flow assay. In this example, the lateral flow assay comprises a reagent to detect, for example, a three-dimensional epitope of an analyte.

According to another example embodiment, the analyte (mixed with the fluid) flows from the input chamber through flow regulation tubing into a first chamber, a lysis chamber. In this chamber, lysis reagents that have been deposited on the walls of the chamber mix with the fluid to lyse the cells, releasing the analyte into the fluid. The analyte then exits the first chamber by flowing through flow regulation tubing into a second chamber, where it undergoes precipitation by mixing with precipitation reagents that have been deposited into the second chamber. Once the analyte has been precipitated, the analyte flows via flow regulation tubing into the sample delivery array for distribution into the lateral flow assay. In this example, the lateral flow assay comprises a reagent to detect, for example, the analyte.

According to another example embodiment, the analyte (mixed with the fluid) flows from the input chamber through flow regulation tubing into a first chamber, a deglycosylation chamber. In this chamber, deglycosylation reagents that have been deposited on the walls of the chamber mix with the fluid to remove glycosylation groups from an analyte, e.g., a protein. The analyte flows via flow regulation tubing into the sample delivery array for distribution into the lateral flow assay. For example, the lateral flow assay comprises a reagent to detect, for example, a deglycosylated protein.

According to another example embodiment, the analyte (mixed with the fluid) flows from the input chamber through flow regulation tubing into a first chamber, a lysis chamber. In this chamber, lysis reagents that have been deposited on the walls of the chamber mix with the fluid to lyse the cells, releasing the analyte into the fluid. The analyte then exits the first chamber by flowing through flow regulation tubing into a second chamber, where it undergoes deglycosylation by mixing with deglycosylation reagents that have been deposited into the second chamber. Once the analyte has been deglycosylated, the analyte flows via flow regulation tubing into the sample delivery array for distribution into the lateral flow assay, In this example, the lateral flow assay comprises a reagent to detect, for example, a deglycoslyated protein.

According to another example embodiment, the analyte (mixed with the fluid) flows from the input chamber through flow regulation tubing into a first chamber, a deglycosylation chamber, where it undergoes deglycosylation by mixing with deglycosylation reagents that have been deposited into the first chamber. Once the analyte has been deglycosylated, the analyte then exits the first chamber by flowing through flow regulation tubing into a second chamber, where it undergoes fragmentation by mixing with fragmentation reagents. Once the analyte has been fragmented, the analyte flows via flow regulation tubing into the sample delivery array for distribution into the lateral flow assay. In this example, the lateral flow assay comprises a reagent to detect, for example, two or more three-dimensional or linear epitopes of an analyte.

Examples of various processing configurations for the second fluid flow pathway include an impurity removal chamber. According to one example, the analyte (mixed with the fluid) flows from the input chamber through flow regulation tubing into an impurity removal chamber. In this chamber, impurity removal reagents that have been deposited on the walls of the chamber mix with the fluid to remove contaminants that could cause false positives.

In some embodiments, the impurity removal agents could be anti-IgG or IgM antibodies to remove unwanted Ig isotypes from plasma. In other embodiments, the impurity removal agents could be cross-reactive antigens to remove antibodies from the sample that could cross react with a similar antigen to the analyte being detected. For example, if the goal is to detect anti-Zika NS1 antibodies, and the patient has previously been infected with or exposed to Dengue, there will be anti-Dengue NS1 antibodies that can cross react with Zika NS1. Therefore, the sample is passed through a chamber containing Dengue NS1 protein to remove the anti-Dengue NS1 antibodies before they arrive in the Zika NS1 detection channel. Then, when a positive response in the anti-Zika NS1 channel is obtained, the result is based on antibodies specific for Zika NS1 due to an anti-Zika response, and not due to a cross reaction from an earlier Dengue infection. A high percentage of individuals with new Zika infections in a Dengue endemic area will have had previous dengue infections and could give false positive tests to Zika. This example applies to many other related pathogen infections that tend to occur together during outbreaks of infections. Thus, a primary improvement of the fluidic device as described herein as compared to other devices is its high accuracy and low false positive rate.

In some embodiments, the first fluid flow pathway will detect one or more types of antigens, and the second fluid flow pathway will detect one or more types of antibodies.

Once the analyte has been purified, the analyte flows via flow regulation tubing into the sample delivery array for distribution into the lateral flow assay for detection of the analyte.

### D. Device Processing Characteristics

As there is a need for increased specificity and sensitivity in a POC or PON (point of need) test, the processing methods described herein are key to obtaining desired functionality.

Removal of impurities, competitor molecules, cross-reactive antibodies, or other cross-reactive antigens before testing for the specific analyte allows improved specificity over existing methods of detection and devices. This purification may occur in the second fluid flow pathway as part of the impurity removal chamber. This processing step removes cross-reactive host antibodies and allows specific antibodies to be detected in patients that have are co-infected or have had past infections with similar pathogens, which is a problem in many endemic areas. This will minimize false positive reactions and increase specificity greatly. Many publications have shown that anti-dengue antibodies can cross react with Zika proteins and vice versa (see, e.g., Priyamvada et al., J. Human antibody responses after dengue virus infection are highly cross-reactive to Zika. virus, Proc Natl Acad Sci USA. (2016) 113: 7852-7857; Dejnirattisai et al,, Dengue virus sero-cross-reactivity drives antibody-dependent enhancement of infection with zika virus, Nat Immunol (2016) 17: 1102-1108, Available online: http://dx.doi.org/10.1038/ni.3515; Harrison S C, Immunogenic cross-talk between dengue and Zika viruses, Nat Immunol (2016), 17: 1010-1012, Available online from: http://dx.doi.org/10.1038/ni.3539; Stealer et al., Specificity, cross-reactivity, and function of antibodies elicited by Zika virus infection, Science, (2016) 353: 8:23-826.)

For example, pretreatment during the lateral flow assay to remove IgG for IgM detection and IgM for IgG detection is also a feature which allows removal of competitor molecules that could block the signal of the other antibody isotype. IgG can bind to the antigen being detected and not allow for IgM to bind giving a False Negative for IgM. Thus, removing IgG for detection of IgM will solve this problem, and vice versa.

Additionally, pretreatment of analytes to allow them to be detected by specific antibodies designed to recognize fragmented analytes, analytes that have been treated to remove glycan modifications (deglycosylation), analytes that have been denatured, or otherwise modified will all increase the signal and improve the signal to noise ratio by creating specific and multiple epitopes for detection. This will increase sensitivity by 200-400% in most cases by providing 2-4×the number of epitopes being recognized by monoclonal antibodies. In addition, by removing glycans and directing antibodies against non-glycosylated epitopes we can increase specificity and decrease False Positives due to cross reactive antigens. Pretreatment allows the most specific detection possible, as well as increases sensitivity.

For example, if one protein is being detected by two monoclonal antibodies, one to detect, and a second to bind it, then there is only one labeled antibody/protein analyte. If the protein is cut into 3 fragments, and a pair of mAbs is used against each fragment, then there are three labeled antibodies/protein analyte, and thus, a 3× (300%) higher signal for that analyte.

Additionally, the device is easy to use and the results may be read from a CLIA Waiver eligible device. These devices are designed to be used in a natural remote setting by both medical personnel and/or other non-medical personnel after receiving instructions on device operation.

Having a design of direct flow through chambers and channels without valves allows for reliable operation under various operating conditions and without the danger of faulty valves or the need for external power sources. This feature allows the POC or PON device to be used globally in remote and developed areas of the globe.

Having a design without resistors, heaters/coolers, power supplies, pumps, detectors, or voltmeters not only results in lower cost, ease of use and independence from complex technical issues, but also allows the device to be deployed in remote regions wherein power supplies may not be available.

### E. Flow Regulation Tubes

The ability to vary and/or determine, the optimal flow regulation tubes is based on known physics and fluid dynamic engineering principles. For example, there are many good formulas and estimates that can be made for calculating the diameter, curves and length of the flow regulation tubes between the chambers based on the fluid's viscosity and whether the fluid will be M laminar vs. turbulent flow. As described herein, blood and plasma will be assumed to be in a laminar flow due to the high viscosity of the fluid along with the slow flow rate. This slow flow rate is important to avoid creating “foam” from the blood as well as allowing time for the sample to incubate in the different chambers.

Moreover, it is generally known that blood begins to have problems flowing through tubes at very small diameters (<100 um). Therefore. in preferred embodiments, flow regulation tubes will be approximately 1-3 mm, 1-4 mm, 1-5 mm, 2-4 mm, 2-5 mm, 3-5 mm, 4-5 mm or 1-5 mm in diameter with, preferably, stepwise pressure differentials (for example, a first pressure differential is applied, wait 10 mins, apply second pressure differential, wait 5 mins, apply third pressure differential). In most preferred embodiments, the diameter of the flow regulation tubes will be 1-3 mm. By working with low pressures, turbulent flow and foaming of blood products will be avoided.

Thus, the design of the flow regulation tubes including, curvature, length, and internal diameter can be calculated using standard application of fluid dynamics appropriate for working with blood and plasma. Blood is made up of plasma (55% of volume), red blood cells (45% of volume), white blood cells and platelets (together making up 1% of volume). In preferred embodiments, erythrocytes are removed from entering the device using, for example, a glass fiber filter that binds the erythrocytes, allowing plasma to enter the subsequent chambers and flow regulation tubes. Plasma is made up of 93% water and a series of proteins, inorganic compounds, lipids and glucose. This makeup causes plasma to behave like a Newtonian fluid with a viscosity of 1.2×10⁻³Pa·s. It will follow laminar flow principles in our device due to the slow flow rate, viscosity and small tube diameter. All of these characteristics of the fluid dynamics with relation to the flow regulation tubes contained in the device as described herein, make it so that we can use the Poiseuille equation to estimate the length and diameter of our flow regulation tubes based on our desired flow rate in microliters/second under specific pressure differentials. See, for example, Pritchard P J. Fox and McDonald's Introduction to Fluid Mechanics, 8th Edition, John Wiley & Sons, 2011 (hereby incorporated by reference in its entirety). These well-known equations will help guide the number (including the maximum number) of processing chambers needed, and the time the analyte (contained within the plasma) needs to be in a given processing chamber.

Further, in the described system, turbulent flow will not be an issue, again due to our small tube diameters (e.g., ˜1 mm-3 mm), low fluid flow velocities, and viscosity. We can readily determine that our Reynold's number is well below the 2000 threshold for transition from laminar flow to turbulence and we do not envision any applications where our Reynold's number will approach or surpass the threshold of 2000. Thus, relying on well-known principles for example, that if we decrease a tube's radius by 5%, we will decrease the flow by 19%, and on the other hand, if we double the radius of the flow regulation tube, we will increase the flow rate by 16 will hold in our system (flow rate increases with r⁴, the fourth power of the radius). Lastly, the flow rate is inversely proportional to the length of the flow regulation tube. Thus, in conclusion, the design of the chambers and flow regulation tubes can be calculated based on these principles as described for example in Pritchard et al. for each specific application.

In some cases where whole blood (with red blood cells) is processed in the device, models can be applied which are designed specifically for dealing with blood flow through small curved tubes such as described in the art. See, for example, Wang C Y and Bassingthwaighte J B. “Blood flow in small curved tubes” J Biomech Eng 2003; 125: 910-913. If needed, however it is likely that the effects will be minimal due to the short length, relatively small bore size, low pressure differential and viscosity.

### F. Fabrication of Pressure Driven Fluidics Device

In some embodiments, the fluidic device has the following components: (1) a top casing or housing with an input port and readout window, (2) a bottom casing or housing with chambers, channels (e.g., for receiving the lateral flow strip), a sample delivery array, lateral flow strips, and tubes, and (3) lateral flow components (e.g., sample pads, lateral flow strips (including control bands, readout bands, conjugate pads. pretreatment reagents and wicks) and (4) processing reagents for the fluid flow pathways. Additionally, a syringe, collection tube, or other component comprising the analyte mixed with fluid is configured to connect to the inlet chamber in order to introduce the analyte into the inlet chamber. Each component is described in additional detail as follows.

In some embodiments, a twist top syringe with a coupling head for attachment to the inlet chamber 305 on the device is produced, this syringe is used for introducing the analyte and fluid (e.g., also referred to as a running buffer) into the device. The syringe may be made of a plastic material such as polypropylene (barrel) and polyethylene (plunger). Both components are unreactive towards most chemicals, and in general, any material that is unreactive is suitable. Additionally, the syringe will be marked with specific indicators informing the operator how far to depress the plunger at each step during the operation of the device.

In some embodiments, the top casing may be formed of plastic using, for example, a plastic molding device. The plastic may be opaque or transparent. The plastic molding device creates a casing having an input port 305 for connecting a syringe to the inlet chamber and a readout window (to which transparent glass, plastic or other transparent material can be attached) to allow the results of the analyte detection bands to be viewed while the top casing is attached to the bottom casing. Any material that is generally unreactive is suitable for forming the top housing.

The bottom casing houses the components for the pressure-driven component and the lateral flow driven component and may also be formed of plastic using, for example, a plastic molding device. Any material that is generally unreactive is suitable for forming the bottom housing. The bottom housing may contain fixtures or other indicated locations for attachment of chambers, delivery arrays, lateral flow strips, and any other component needed for the device.

In general, the device comprises a cover that snaps into place and seals the readout window creating a closed system. Thus, the chambers can be filled with appropriate dry chemicals or micro/nanobeads embedded or coated with specific chemicals, proteins or antibodies for any of the processing procedures outlined above. In some embodiments, a desiccant may be incorporated into the device to prevent moisture from being absorbed into the dry reagents, and the assembled device will be sealed in an airtight pouch.

In some embodiments, one or more of the chambers and sample delivery array are integrated with the housing, e.g., formed from the same material and in the same manufacturing process as the bottom housing. In this scenario, other components are then added to complete assembly of the device, including flow regulation tubing to connect chambers and conjugate pads, lateral flow strips, and wicks to facilitate lateral flow of the processed analyte.

In other embodiments, one or more of the sample recipient well, chambers, and sample delivery array are formed separate from the housing, e.g., with the chambers formed separately as discrete components and then affixed to the bottom casing. Other components are then added to complete assembly of the device, including flow regulation tubing to connect chambers and conjugate pads, lateral flow strips, and wicks to facilitate lateral flow of the processed analyte.

To form the lateral flow test strip, the following series of steps may be performed. In general, conjugate pads and wicks (Whatman 470 or Ahlstrom 222) are discrete components that may be purchased from (Sigma-Aldrich Co. LLC. and Ahlstrom Corp.), and then affixed to the lateral flow component of the device. Then, in a stepwise manner, reagents are printed or sprayed onto the lateral flow strip. For example, in one embodiment, the conjugate pad (e.g., Ahlstrom 8950 or Millipore GFDX) is printed or sprayed with reagents (e.g., reagents for detection such as conjugated reagents, antibodies, recombinant antigens, etc.); then, reagents are deposited onto the nitrocellulose membrane (e.g., for a sample pre-treatment area, control lines, and for test lines). Once reagents have been deposited, the lateral flow strips are cut into strips of 4-5 mm width for attachment to the bottom housing, e.g., a self-adhesive card (Lohmann).

Reagents, detection molecules, and labels for readout of positive or negative results and the control band may be incorporated into the device as described herein. The reagents used in the device will be manufactured separately. For example, reagents for the first fluid flow pathway and the second fluid flow pathway may be printed, sprayed, freeze-dried, lyophilized, or deposited by any other known method into the chambers. These reagents include: neutral buffers, detergents, surfactants, acids, bases, and enzymes, monoclonal or polyclonal antibodies, or proteins.

Reagents for the lateral flow strip, including the pretreatment region and analyte readout bands may be printed, sprayed, freeze-dried, lyophilized, etc. onto the test strip. These reagents may include antibodies and/or antigens labeled with gold nanoparticles or latex nanoparticles, monoclonal or polyclonal antibodies, recombinant antigens, purified proteins, and controls. These reagents are bound to the nitrocellulose membranes or conjugate pads.

In other embodiments, reagents such as diluents, stabilizers, etc. are contained in the running buffers (e.g., the fluid from the syringe used to propel the analyte through the pressure-driven component of the device.

Once assembled, the device is packaged in an airtight pouch with a desiccant. All manufacturing steps are performed under ISO (certified to ISO 13485-2003) and GMP compliant conditions. These components of the housing will be produced using plastics such as polypropylene and a polyethylene either by 3D printing, and/or by injection molding manufacturing, but always using FDA approved materials and an ISO certified manufacturer.

### G. Coating of Reagents into Chambers

In one preferred method, the reagent can be integrated or placed onto the interior surface of the chamber by a pipetting robot (e.g., a Hamilton robot—microlab nimbus, Thermo scientific hydra II, etc.), a sprayer (e.g., a ZETA precision dispenser, DCI, Biodot airjet dispenser, X1010, electrospraying, etc.), a piezoelectric inkjet printer (e.g., a Microdrop nanojet, a Gesim nanoplotter, a Tecan HP D300 digital dispenser, a picoliter dispenser topspot, a dimatric DMP 2800, a Xerox phaser 8560N color printer), or by contact printing or microcontact printing. Contact printing brings a delivery component, such as stamp, pin or other structure, into physical contact with the surface for which the reagent is to be deposited.

Depositing reagents may generally fall into two categories: non-contact or contact methods. Non-contact methods include pipetting robots, sprayers, and piezoelectric inkjet printers, and can deposit multiple reagents to the same (or nearby) location without cross contamination. For example, using piezoelectric inkjet printers, picoliter to nanoliter volumes can be spotted onto specific chamber locations.

For sprayers, such as electrosprayers, solvents with low vapor pressure can be utilized. In sonic embodiments, organic solvents are utilized, and the solvent evaporates once the reagent is deposited on the surface. In other embodiments, water may be used as a solvent, and evaporated or lyophilized after deposition. In some embodiments, noncontact methods may be used, e.g., as sprayer, to deposit reagents of the readout hand (e.g., capture antibodies).

Thus, in general, the reagents are either dry-deposited within the chamber, or lyophilized from a liquid within the chambers. Alternatively, the reagents may be added as micro/nano beads embedded or coated (covalent or non-covalently) with the reagents of interest. Then, when the fluid with sample/analyte(s) arrives, the reagents will dissolve, and processing of the analytes by the reagents will occur.

### H. Analytes

Mosquito borne illnesses include but are not limited to malaria, dengue fever, West Nile virus, chikungunya, yellow fever, filariasis, Japanese encephalitis, Saint Louis encephalitis, Western equine encephalitis, Eastern equine encephalitis, Venezuelan equine encephalitis, La Crosse encephalitis and Zika fever.

Tumor antigens include, but are not limited to MAGE-A1, MAGE-A2, MAGE-A3, MAGE-A4, MAGE-A5, MAGE-A6, MAGE-A7, MAGE-A8, MAGE-A9,MAGE-A10, MAGE-A11, MAGE-A12, GAGE-1, GAGE-2, GAGE-3, GAGE-4, GAGE-5, GAGE-6, GAGE-7, GAGE-8, GAGE-9, BAGE, BAGE-1, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, MAGE-C3, MAGE-C4, MAGE-C5, NY-ESO-I, LAGE-I, SSX-1, SSX-2 (HOM-MEL-40), SSX-3, SSX-4, SSX-5, SCP-I and XAGE, melanocyte differentiation antigens, p53, ras, p21ras, CEA, MUCI, PMSA, PSA, tyrosinase, Melan-A, MART-1, gp100, gp75, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-2, and 3, neo-PAP, myosin class I, OS-9, pml-RAR alpha fusion protein, PTPRK, K-ras, N-ras, Triosephosphate isomerase, GnT-V, Herv-K-mel, NA-88, SP17, and TRP2-Int2, MART-1, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, alpha.-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\170K, NY-CO-1, RCAS1, SDCCAG16, TA-90 (Mac-2 binding protein\cyclophilin C-associated protein), TAAL6, TAG72, TLP, TPS, tyrosinase related proteins, TRP-1, TRP-2, TRP-2/INT2, or mesothelin, HER-2/neu, 707-AP, AFP, ART-4, p190 minor bcr-abl, CAMEL, CAP-1, CAP-2, CDC27, CT, Cyp-B, DAM, ELF2M, GAGE, RAGE, HLA-A*0201, HLA-A*1101, HLA-A*0201-R1701, HLA-B*0702, HPV-E7, HAST-2, hTERT (or hTRT), iCE, LAGE, LDLR/FUT, MC1R, myosine/m, MUC1, NA88-A, NY-ESO-1, PRAME, PSA-1, PSA-2, PSA-3, prostate-specific membrane antigen (PSMA), PSM, RU1 or RU2, SAGE, SART-1 or SART-3, TEL/AML1, TPI/m, and WT1, adenosine deaminase-binding protein (ADAbp), FAP, cyclophlin b, CRC C017-1A/GA733, AML1, CD20, alpha-fetoprotein, E-cadherin, alpha-catenin, beta-catenin, gamma-catenin, p120ctn, adenomatous polyposis coli protein (APC), fodrin, Connexin 37, Ig-idiotype, p15, GM2 ganglioside, GD2 ganglioside, Smad family of tumor antigens, Imp-1, PIA, EBV-encoded nuclear antigen (EBNA)-1, brain glycogen phosphorylase, CT-7, and c-erhB-2.

Tick borne illnesses include but are not limited to Anaplasmosis, Babesiosis, Borrelia mayonii, Borrelia miyamotoi, Colorado tick fever, Ehrlichiosis, Heartland virus. Lyme disease, Powassan disease, Rickettsia parkeri rickettsiosis, Rocky mountain spotted fever (RMSF), Southern tick-associated rash illness (STARI), Tickborne relapsing fever (TBRF), Tularemia, and 364D rickettsiosis.

Non-communicable diseases include but are not limited to cardiovascular disease (e.g., coronary heart disease, stroke, etc.), cancer, chronic respiratory disease, diabetes, chronic neurologic disorders e.g., Alzheimer's disease, dementias), and arthritis/musculoskeletal diseases.

Bacterial illnesses include but are not limited to Streptococcal bacteria, Escherichia coli, Salmonella, Helicobacter pylori, Neisseria gonorrhoeae, Neisseria meningitides, Staphylococcus aureus, Streptococcal bacteria, Actinomycoses and nocardiosis, Anthrax, Brucellosis, Buruli ulcer, Capnocytophaga, Elizabethkingia, Glanders (Burkholderia mallei), Hansen's disease (Leprosy), Leptospirosis, Melioidosis (Burkholderia pseudomallei), Pasteurella sp. infections, and Rat-Bite fever.

Parasitic illnesses include but are not limited to protozoan (e.g., leishmaniasis, chagas disease, malaria, toxoplasmosis, etc.) and helminths (e.g., tapeworms, flukes, roundworms, etc.).

Fungal illnesses include but are not limited to Aspergillosis, Blastomycosis, Candidiasis, Coccidioidomycosis, C. neoformans Infection. C. gattii Infection, Fungal Eye Infections, Fungal Nail Infections, Histoplasmosis, Mucormycosis, Pneumocystis pneumonia, Ringworm, and Sporotrichosis.

Antibodies against allergens responsible for allergies to food allergies (e.g., peanuts, tree nuts, crustacean shellfish, fish, wheat (gluten), milk, egg, soybeans, etc.) or airborne allergies (pollen, mold, dust mite, animals, etc.).

Cancers include, but are not limited to the cancer is a leukemia, adenocarcinoma, sarcoma, skin cancer, melanoma, bladder cancer, brain cancer, breast cancer, uterine cancer, ovarian cancer, prostate cancer, lung cancer, colorectal cancer, cervical cancer, liver cancer, head and neck cancer, esophageal cancer, pancreas cancer, pancreatic ductal adenocarcinoma (PDA), renal cancer, stomach cancer, multiple myeloma and/or cerebral cancer.

### EXAMPLES

In general, present invention embodiments include a pressure driven component and a lateral flow driven component. A syringe, in fluid communication with the pressure driven component, introduces a fluid containing the analyte into the device, and provides force to propel the fluid through the pressure driven component. The pressure driven component comprises at least two fluid flow pathways, with the first fluid flow pathway and the second fluid flow pathway comprising different processing steps, as described herein. Each fluid flow pathway comprises one or more chambers, wherein each chamber is configured to perform one or more processing steps and wherein the fluid enters and exits the chamber via flow regulation tubing. Each chamber contains one or more reagents for processing the analyte. Once the analyte has been processed, the analyte travels through a sample delivery array, where it is distributed into the lateral flow component comprising a plurality of channels. Each channel contains a lateral flow strip. The analyte travels down the lateral flow strip by capillary action, optionally undergoing further processing, such as pre-treatment, before contacting analyte readout bands for detection. The examples presented herein are not intended to be limiting. Example embodiments are provided as follows.

### Example 1: Methods of Using Device

In a first example embodiment, a blood sample is obtained from a patient. The blood sample is mixed with a diluent, such as PBS buffer or any other non-reactive buffer, and a syringe is then filled with the fluid comprising the analyte.

The syringe is attached to the top casing of the device. Pressure is applied to the syringe to propel the fluid containing the analyte through the first fluid flow pathway and the second fluid flow pathway. During the first fluid flow pathway, the blood cells are lysed and the analyte is released into the fluid. The analyte is then denatured, and is distributed to the lateral flow strip for detection. For example, the lateral flow test strip may detect a linear epitope of an infectious pathogen protein.

During the second fluid flow pathway, impurities are removed from the fluid, such as cross-reactive antibodies. Analytes flow through the second fluid flow pathway and are distributed to the lateral flow strip for detection. In this example, the lateral flow test strip may detect antibodies against an infectious pathogen protein. Thus, in some embodiments, the second fluid flow pathway will not denature, lyse, or deglycosylate, but rather, will detect host responses against a pathogen (e.g., by detecting antibodies against a. pathogen or allergen, by detecting secreted molecules, or by detecting surface or extracellular markers) Accordingly, a positive diagnosis may be made.

In a second example embodiment, a blood sample is obtained from a patient for multiplex differential diagnosis of viral diseases (Dengue vs. Zika). The blood sample is mixed with a diluent, such as PBS buffer or any other non-reactive buffer, and a syringe is then filled with the fluid comprising analyte(s).

The syringe is attached to the top casing of the device. Pressure is applied to the syringe to propel the fluid containing analytes through the first fluid flow pathway and the second fluid flow pathway. During the first fluid flow pathway, the blood cells are lysed and the analytes are released into the fluid. The analytes are then fragmented, and distributed to the delivery array and to lateral flow strips for detection. For example, one lateral flow test strip for detection of Dengue may detect three epitopes (e.g., one epitope on each fragment) of a Dengue protein A in one detection band, and detect three epitopes (e.g., one epitope on each fragment) of another Dengue protein B in another detection band. Another lateral flow test strip for detection of Zika, may detect three epitopes (e.g., one epitope on each fragment) of a Zika protein A in one detection band, and detect three epitopes (one epitope on each fragment) of another Zika protein B in another detection band.

In general, each detection band may detect a single type of analyte or may detect multiple types of analytes. Embodiments of the invention include both configurations.

During the second fluid flow pathway, impurities are removed from the fluid, such as IgG antibodies or IgM antibodies. Analytes (in this example, anti-Zika or anti-Dengue antibodies) flow through the second fluid flow pathway and are distributed to the delivery array arid to lateral flow strips for detection. In this example, one lateral flow test strip has Zika antigens in the pre-treatment zone, and will detect anti-Dengue antibodies in a detection hand using Dengue antigens. Another lateral flow test strip has Dengue antigens in the pretreatment zone, and will detect anti-Zika antibodies in a detection band using Zika antigens.

Accordingly, a highly sensitive positive diagnosis for both Dengue and/or Zika can be made, as well as determination of active versus past infection with either virus, e.g., through detection (positive or negative) of anti-Dengue antibodies or anti-Zika antibodies.

In a third example embodiment, a blood sample is obtained from a patient for multiplex detection of a series of cancer indicating analytes. The blood sample is mixed with a diluent, such as PBS buffer or any other non-reactive buffer, and a syringe is then filled with the fluid comprising the analytes.

The syringe is attached to the top casing of the device. Pressure is applied to the syringe to propel the fluid containing the analytes through the first fluid flow pathway and the second fluid flow pathway. During the first fluid flow pathway, analytes are deglycosylated in one chamber and fragmented in another chamber. The fragmented analytes are then distributed to the delivery array and to a lateral flow strip for detection. For example, one lateral flow test strip may detect two epitopes (one epitope on each of two fragments) of a cancer specific antigen in one detection band, and may detect three epitopes (one epitope on each of three fragments) of another cancer specific antigen in another detection band.

During the second fluid flow pathway, analytes are maintained in their native state. Analytes (in this example, patient inflammation response proteins) flow through the second fluid flow pathway and are distributed to the delivery array and to a lateral flow strips for detection. In this example, one lateral flow test strip will detect inflammation response protein A, another will detect inflammation response protein B, and still another will detect a third inflammation response protein C, each in distinct detection bands. In general, each detection band may detect a single type of analyte or may detect multiple types of analytes. Embodiments of the invention include both cases. Accordingly, a positive diagnosis for the presence of a specific cancer can be made.

In a fourth example embodiment, a urine sample is obtained from a patient for multiplex detection of a series of asthma related analytes. The urine sample is mixed with a diluent, such as PBS buffer or any other non-reactive buffer, and a syringe is then filled with the fluid comprising the analytes.

The syringe is attached to the top casing of the device. Pressure is applied to the syringe to propel the fluid containing the analytes through the first fluid flow pathway and the second fluid flow pathway. During the first fluid flow pathway, analytes are fragmented. The fragmented analytes are then distributed to the delivery array and to a lateral flow strip for detection. For example, one lateral flow test strip for detection of two epitopes (one epitope on each of two fragments of an inflammatory marker in one detection band.

During the second fluid flow pathway, analytes are maintained in their native state. Analyte flows through the second fluid flow pathway and is distributed to the delivery array and to a lateral flow strip for detection. in this example, one lateral flow test strip will detect a non-protein marker such as leukotriene E4 in a specific band. In general, each detection band may detect a single type of analyte or may detect multiple types of analytes. Embodiments of the invention include both cases.

### Example 2: Processing Removes Dengue Patient False Zika Positive Signal, Increases Sensitivity to Detect Host Anti-Dengue Virus Antibody Response, and Increases Sensitivity to Detect Dengue Virus

One important application of our fluidic device is the detection of mosquito-home viruses that circulate simultaneously in the population. Three such viruses of global impact are Dengue, Zika and Chikungunya, each of which can lead to outbreaks with overlapping and diverse clinical characteristics (fever, rash, joint pain) making it impossible for the health post worker, clinician, or public health professional to accurately diagnose a patient based on clinical signs and symptoms alone. Thus, a field use point of care diagnostic device designed to be used by anyone, anywhere, without requiring electrical power that accurately and quickly detects infections with these three viruses is a critical need for global public health.

However, problems exist with the currently used diagnostic tests detecting mosquito-borne viruses namely a high rate of false positives and/or low sensitivity. False positives occur when a test detects antibodies from an infected individual that are capable of binding (cross-reacting) to more than one virally derived antigen. Cross-reactivity is frequently observed in patients infected with Dengue virus. Here, the patient's antibodies generated by a Dengue infection are capable of cross-reacting to NS1 protein generated from Zika virus. As described herein, the processing of the biological sample as described herein solves this problem by detecting both the presence of the virus (detection of proteins from Dengue, Zika and Chikungunya) as well as the detection of the specific host response against these viruses (antibodies against the viruses). Moreover, processing the biological sample as described herein can be used by anyone, anywhere, free from outside energy sources or complex readers. A test that provides rapid, accurate and sensitive detection of infections with Dengue, Zika and Chikungunya in the field would revolutionize patient care allowing for health professionals to immediately triage patients based on with which virus(es) an individual is infected.

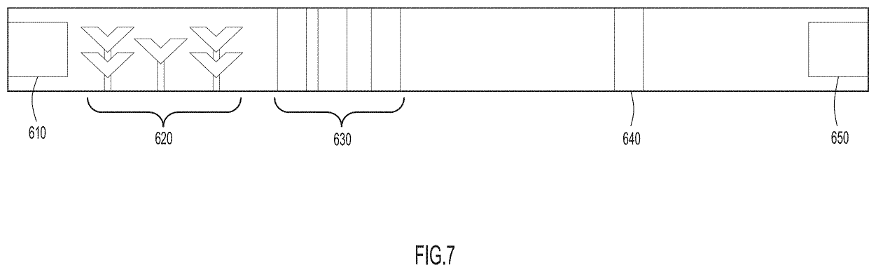

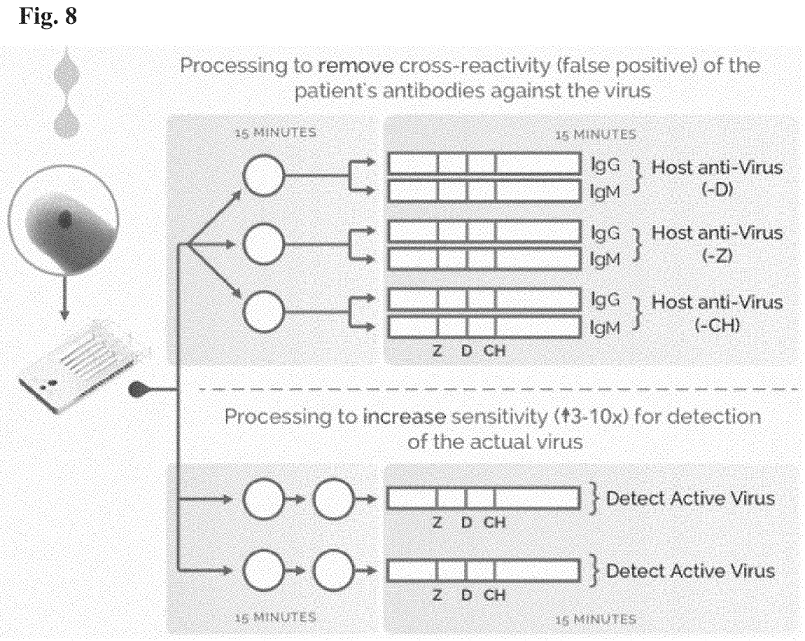

Our first series of studies designed to test our processing technology have shown that we achieve greater specificity (removing false positives), and a ˜200-500% increase in sensitivity compared to unprocessed samples using blood samples from patients naturally infected with Dengue in the acute phase of disease. An overall schematic for potential application of the processing steps towards the simultaneous detection of Dengue, Zika and Chikungunya is shown in FIG. 8.

Specifically, a blood sample can be applied to a pressure driven fluidics device as described herein via the use of a blood collection tube (not shown). The sample is then channeled via pressure driven force into the processing chambers for either; 1) removal of host antibody cross-reactivity (false positives) against each of the viruses (top panel) and subsequent detection of host anti-virus IgG and IgM antibodies, and 2) increase in virus protein availability for detection of virus particles (NS1 in this example) using our processing methods. As shown below, the processing methods described herein:
1. Identify false Zika positive tests caused by a cross-reactivity of Dengue infected patient antibodies with the Zika virus. Removing the false positive signal greatly increasing specificity (this high rate of false positives for Zika was identified by the FDA in a recent alert for the “ZIKV Detect” kit).
2. Increase by an average ˜200% the sensitivity of anti-Dengue IgM antibody detection.
3. Increase by an average —400% the sensitivity of detecting the presence of the actual virus in a patient's blood via detection of viral NS1.

All of the processing methods were performed using micro-columns (Mobicol-mini columns), tubes (250 ul microtubes) and plate wells (96 well plates) in volumes (45 ul-100 ul) and using procedures that are independent of any external electrical supply or laboratory equipment to mimic the processes that will take place in processing chambers in the pressure driven fluidics device described herein. All reactions to detect human antibodies against viral proteins were performed using lateral flow strips and running buffer in conjunction with anti-human IgM or anti-human IgG antibodies conjugated with gold nano-particles.

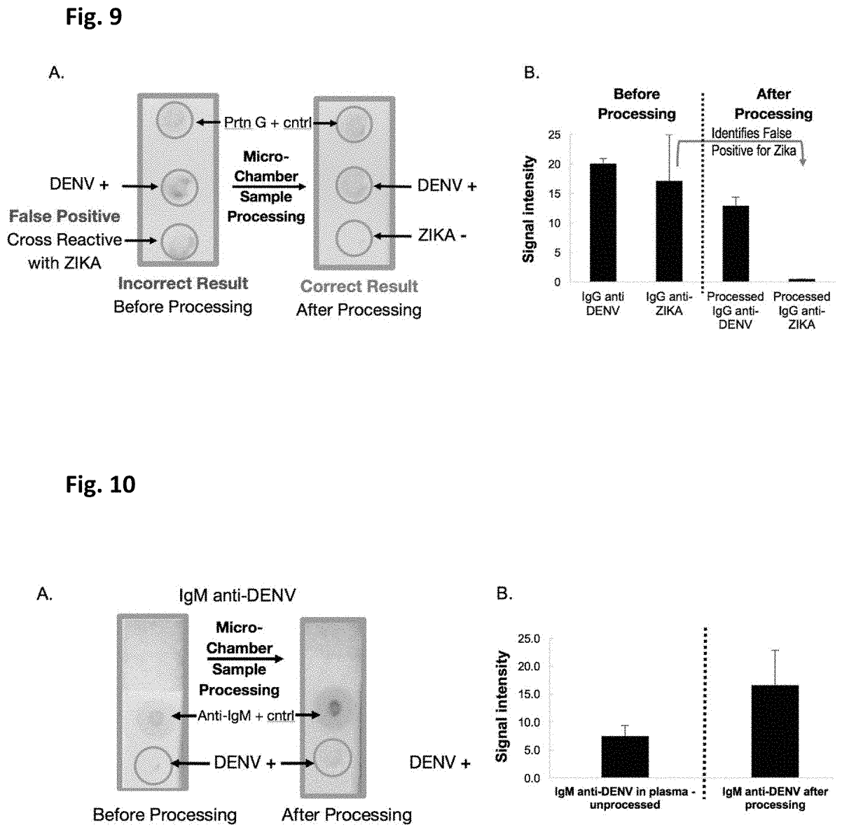

Sample preparation and data processing: In the following experiments, all Dengue patient plasma were obtained from patients in the acute phase of disease (i.e., between 1-7 days of symptoms onset) and maintained frozen at −80° C. until use. All samples were confirmed DENY positive with PCR and ELISA. The tests for detection of human anti-Dengue NS1 IgG or IgM were performed using three plasma pools of four different individuals each diluted 1:10 with running buffer just before running on the test strip (45 ul/run, see above). The experiments for detection of soluble Dengue NS1 in the human plasma were performed using three pools of two different individuals each diluted 1:2 in running buffer just before running the test strip (40 ul/run, as further described below). The experiments for detection of NS1 released from human white blood cells from Dengue patients were performed using 8 ul of lysate derived from lysis of cells from three separate individuals. Here white blood cells were washed, counted and resuspended to 1.5-1.9×10⁶ cells/ml in lysis buffer replicating the window of cell concentration in whole blood. After a 5 minute incubation at RT, a 1:5 dilution of lysis solution was made in running buffer and applied to the lateral flow strip treated with the anti-Dengue NS1 capture antibody, followed by detection as described above. The strips were read visually (FIGS. 9-12) and also images taken with a digital camera followed by determination of spot intensity using ImageJ (NIH). Equivalent areas were delineated around spots of interest for which Image) returns a mean grey value. The intensity of a given positive spot was then calculated by subtracting the background from the same strip. These values were then plotted as mean +/−standard deviation as an estimate of the fold or percent increase or decrease between processed vs. processed samples as seen in FIGS. 9-12.

Results: Processing Decreases False Positives. To test the ability of our processing to remove a false Zika positive IgG response we first tested Dengue patient plasma for a positive signal against Dengue NS1 and a false positive signal against Zika NS1. Lateral flow strips were spotted with Dengue NS1, Zika NS1, and a positive control spot of protein G. Three pools of plasma, each containing four different Dengue patients, were made and run on lateral flow strips followed by detection of IgG anti-Dengue NS1 or anti-Zika NS1. As can be seen in FIG. 9A-B for detection of host IgG anti-viral particles (FIG. 9A), the patient plasma pool shows a positive signal for IgG antibodies against Zika NS1 (false positive) and Dengue NS1 (Left lateral flow strip image, FIG. 9A). The average reactivity of all three pools following calculation of the spat intensity is shown in FIG. 98 (left bars, “before processing”) demonstrating the false positive anti-Zika IgG response. Other strips were used for detection of IgG anti-Dengue NS1 or anti-Zika NS1 following micro-processing during which the plasma was pre-incubated with Zika NS1 for 15 mins at room temperature, followed by flow through into microwells for running on the lateral flow strips. As can be seen in FIG. 9A-B, the processing was able to remove the false Zika positive signal in the Dengue infected patients giving the correct result of Dengue positive (FIG. 9A, right panel “After Processing”). The average spot intensity of all three pools also showed the dramatic removal of the false Zika positive signal (FIG. 9B, right bars “After Processing”).

In this example, three pools of four patient plasmas were run on lateral flow strips spotted with the positive control protein G, Dengue NS1, and Zika NS1 (FIG. 9A, from top to bottom). Before processing a positive signal is seen for both IgG against Dengue and a false positive (due to cross reactivity) against Zika. (FIG. 9A, left strip). After pre-processing with soluble Zika NS1 for 15 mins, the correct result is seen, with the removal of the false positive IgG anti-Zika. response, and continuation of the IgG anti-Dengue response (FIG. 9A, right strip). FIG. 9B shows the average and standard deviation of signal intensity using three Dengue patient sample pools before processing (left bar) and after processing (right bar), and the resulting elimination of the false Zika positive response. Intensity was determined as described above.

As can be seen, the processing dramatically reduced the false positive signal, showing the utility of the method for removing the false positive signal without any sophisticated equipment and over a treatment period of only 15 minutes.

Results: Processing Increases Sensitivity Of Detection Of Dengue Patient IgM Against DENV Protein NS1. IgM detection can be inhibited due IgG antibody competing for binding to the target. To test the ability of sample processing to increase sensitivity for detection of IgM against viral proteins the pooled Dengue infected patient samples were processed to remove IgG using micro-flow through protein G conjugated beads by pressure alone (no sophisticated equipment or energy source, just pressure from a syringe). Again, three pools of four Dengue patient plasma samples were processed by flow through incubation with protein G conjugated agarose beads (to remove IgG) and then run on lateral flow strips for detection of IgM anti-DENV NS1. FIG. 10A shows the IgM anti-DENV response before processing (left strip, weak signal), and after processing to remove IgG (right strip, stronger signal). The average and S.D. of signal intensity of the three pools is shown in FIG. 10B, where it is clearly seen that the processing increases the sensitivity of detection by approximately 2.5 fold (fold increase in IgM signal range: 0.95-2.8. The intensity was determined as described above.

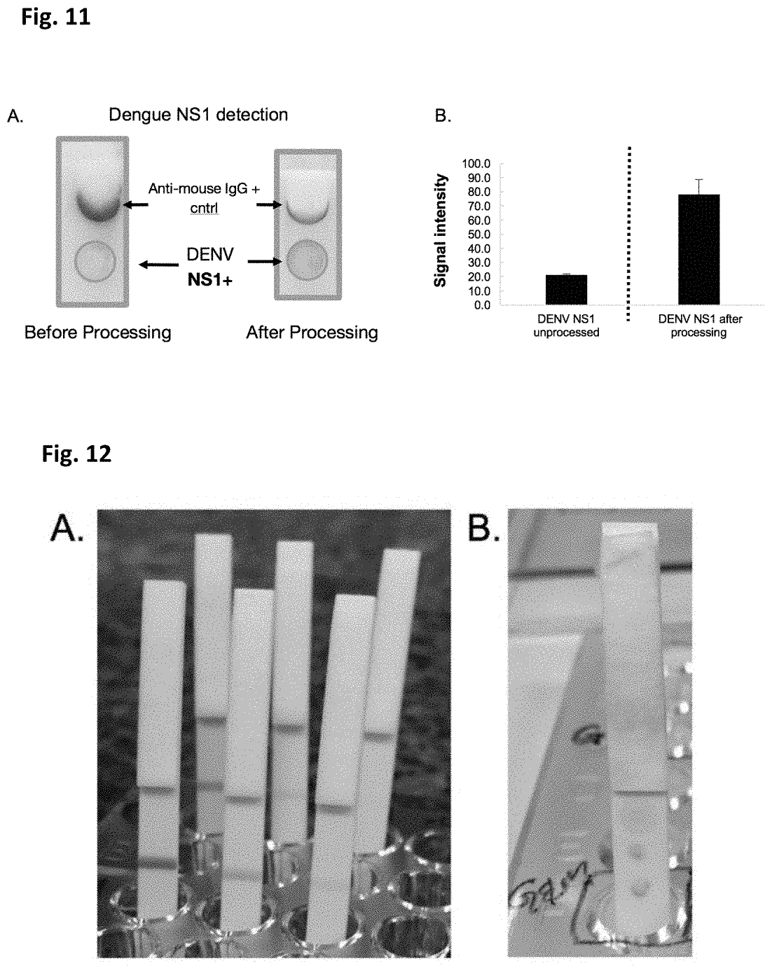

Results: Micro-Processing Increases Detection of Virus Particles from Dengue Infected Patient Samples. Viral diseases lead to infection of the body's cells and proteins released from infected cells can be detected in the blood and blood products of infected individuals. These proteins can in turn be detected using diagnostic tools. One such protein, NS1 from Dengue (an analog of NS1 is also present in Zika and Chikungunya), is detectable in the blood and plasma from infected individuals, however, it is often at low levels and begins to decline shortly after infection (˜5-7 days) due to an increased immune response which starts to clear the infection. Importantly, there is a “reserve” of NS1 in the blood that is ignored and lost in currently used blood exams. Many blood cells are infected with Dengue and thus contain NS1 within the cells. Thus, to test the ability of blood processing as described herein to liberate cellular NS1 for later detection, blood derived white blood cells from Dengue infected individuals (in the acute phase of infection) were processed by lysing white blood cells and running the lysate on the same lateral flow detection used to detect NS1 in the plasma. NS1 is detected using an anti-Dengue NS1 capture antibody and an anti-Dengue N1 detection antibody conjugated with nano-gold particles. Lysis and detection of DENY NS1 in the lysate leads to dramatic increase in the signal intensity and availability of NS1 target for detection in solution. FIGS. 11A-B. While NS1 is detected in the plasma (FIG. 11A, left strip), the intensity of NS1 detection from the lysate of cells is greater and leads to a dramatic increase in signal intensity (FIG. 11A, right strip). The average and SD of three individual Dengue infected patient samples is shown in FIG. 11B for unprocessed plasma (left bar) and after processing of blood cells to liberate NS1 (right bar), leading to a 23.50-530% increase in whole blood adjusted signal intensity over plasma alone.

Thus, the above processing steps using micro-chambers (tubes, wells and columns), with no outside energy source or complex laboratory equipment, leads to increased specificity and sensitivity of Dengue virus detection from blood of acutely infected individuals. This same procedure can easily be applied to detection of Zika and Chikungunya, as well as other blood derived analytes.

All lateral flow experiments were performed using strips composed of a nitrocellulose membrane (Millipore HF180) and absorbent wick (Millipore CO48) affixed to an adhesive backing (Millipore MIBA-040). Each strip was composed of a 2 cm detection area followed by 2 cm of absorbent wick. The reagents in 1×PBS (pH 7.4, Sigma-Aldrich) were hound to the nitrocellulose in a volume of 0.5 ul spotted using a pipette and dried at RI for at least 2 hours.

Strips for detection of antibodies against Dengue NS1 and Zika NS1 were prepared as follows. The strips were prepared as mentioned above starting with the positive control spot at the top composed of 1.2 ug/0.5 ul of protein G, followed by 0.9 ug/0.5 ul of Dengue NS1 (Native Antigen Co), and 0.9 ug/10.5 ul of Zika NS1 (Native Antigen Co). The strips for detection of soluble Dengue NS1 in the Dengue patient samples (plasma or lysate from processed cells) were prepared by starting with the positive control spot at the top of 1 ug/0.5 ul of goat anti-mouse IgG, followed by 1 ug/0.5 ul of capture antibody against Dengue NS1 (BBI, #BM404-K2G4).

Following binding of the reagents, the membranes were blocked using 1.6 ml of 1×Meridian blocking buffer (Merdian Life Sciences, #J82300B) in 5 ml conical tubes for 10 mins, followed by three washes with 1.6 ml of 1×PBS pH 7.4 for 5 mins each. The strips were then dried at RT and wicks blotted with Kim Wipes and ready to use after at least 2 hours. The lateral flow assays were always run using passive absorption and wicking of the test sample (45 ul) up the strip by inserting the nitrocellulose end into the solution to be tested (patient plasma+running buffer (Innova Biosciences), or patient cellular lysate+running buffer) in wells of a 96 well plate (FIG. 12). As soon as the sample had run up the strip (˜10 mins), the sample was followed by 45 ul of the detection antibody (anti-human IgG at ⅕ dilution in running buffer of 10 OD stock (Fitzgerald, #103150), anti-human IgM at ⅓ dilution in running buffer of 10 OD stock (Fitzgerald, #103149), or anti-Dengue NS1 at ⅕ dilution of 10 OD (BBI, #BM409-G3D1) for another 10 mins. All were conjugated with InnovaCoat® GOLD nanoparticles as per manufacturer's instructions (Innova Biosciences, #280-0000). A final chase of 20 ul of running buffer was performed for 10 mins. The conjugation was confirmed using Conjugate Check and Go (Innova Biosciences) strips as per manufacturer's instructions (FIG. 12). The positive reactions were visualized by the naked eye, and in some cases digital photographs were taken and the intensity of the spots determined using Image) (NIH). The intensity of the spots was reported with the background subtracted. FIG. 12 shows the application of some lateral flow experiments as run in these studies.

Specifically, FIG. 12A shows strips used to test the conjugation of anti-human IgM (back row) and anti-Human IgG (front row) at three different dilutions using the Check and Go strips (Innova Biosciences). FIG. 12B shows the running of a strip for detection of human IgG anti-NS1 from a pool of Dengue patient plasmas, detected using anti-human IgG conjugated with nanogold particles, From the bottom up is detection binding of human IgG to Zika NS1, followed by Dengue NS1, and the positive control protein G binding the labeled detection antibody.

1. A pressure driven fluidics device comprising:
(a) an inlet for introducing by manually applied pressure a fluid comprising an analyte into the device;
(b) at least two fluid flow pathways in fluid communication with the inlet, wherein each fluid flow pathway comprises one or more chambers, wherein each chamber is configured to perform one or more different processing steps to process the analyte, and wherein each of the one or more chambers is selected from:
(a) a denaturing chamber comprising one or more denaturing reagents to denature the analyte;
(b) a neutralization chamber comprising one or more neutralization reagents o neutralize the analyte;
(c) a solubilizing chamber comprising one or more solubilizing reagents to solubilize the analyte;
(d) a fragmentation chamber comprising one or more fragmenting or cleavage reagents;
(e) an impurity removal chamber comprising one or more reagents to remove one or more non-analyte molecules from the fluid:
(f) a lysis chamber comprising one or more reagents to lyse cells;
(g) a precipitation chamber comprising one or more reagents to precipitate the analyte:
(h) a deglycosylation chamber comprising one or more reagents to deglycosylate the analyte;
(i) a sample pre-treatment zone for removal of one or more unwanted or competitor molecules from the fluid;
(j) both (f) and (g);
(k) both (a) and (b);
(l) both (f) and (h);
(m) each of (a), (d) and (h);
(n) each of (a), (b), (d) and optionally (h); or
(m) any combination of (a)-(n).
and wherein each fluid flow pathway comprises at least one flow regulation tube; and
(c) at least one lateral flow strip in fluid communication with each fluid flow pathways, wherein the lateral flow strip comprises one or more readout bands configured to detect the differently processed analyte.

2. The device of claim 1, wherein the device further comprises a filter positioned between the inlet and the fluid flow pathway.

3. (canceled)

4. The device of claim 1, wherein the diameter and/or curvature of the flow regulation tube is selected based upon the viscosity of the fluid and/or the desired flow rate.

5. The device of claim 4, wherein the diameter of the flow regulation tub is selected from: (a) 1-3 mm, (b) 1-4 mm, (c) 1-5 mm, (d) 2-4 mm, (e) 2-5 mm, (f) 3-5 mm, (g) 4-5 mm or (h) 1-5 mm.

6. The device of claim 1, wherein fluid flows through the lateral flow strip by capillary action.

7. The device of claim 1, wherein the analyte is further processed on the lateral flow strip prior to contacting the readout bands.

8. The device of claim 1, wherein the fluid is:
(a) mixed with an additional buffer, preferably EDTA or heparin, before introduced into the device;
(b) introduced into the device by a collection tube inserted into the inlet;
(c) introduced into the device by a syringe;
(d) any combination of (a)-(d).

9. The device of claim 1, wherein the fluid is selected from blood, saliva, urine, feces, an environmental sample, or a biological sample comprising a pathogen or molecule(s) associated with the presence of a disease.

10. The device of claim 1, wherein the analyte is selected from a lipid, a carbohydrate, a protein, an organic compound, an inorganic compound, a nucleic acid, or a cancer specific antigen.

11. The device of claim 10, wherein the analyte is an antibody.

12. The device of claim 1, wherein the first flow pathway is capable of detecting a first analyte comprising a protein antigen and the second flow pathway is capable of detecting a second analyte comprising an antibody that binds to the protein antigen.

13. The device of claim 12, wherein the protein antigen is in solution in the fluid.

14. The device of claim 12, wherein the protein antigen is released from a cell in the fluid by the denaturing, lysis, and/or fragmentation chamber.

15. The device of claim 1, wherein the device comprises multiple fluid flow pathways configured to detect and distinguish between Dengue infection, Zika infection and Chikungunya infection.

16. The device of claim 15 comprising:
(a) at least one fluid flow pathway capable of detecting any one of Dengue, Zika or Chikungunya extracellular antigens in the fluid;
(b) at least one fluid flow pathway capable of detecting any one of Dengue, Zika or Chikungunya intracellular antigen in the fluid;
(c) at least one fluid flow pathway capable of detecting either a IgM or IgG antibody that can bind to any one of Dengue, Zika or Chikungunya antigen, removing Dengue false positives;
(d) at least one fluid flow pathway capable of detecting either a IgM or IgG antibody that can bind to any one of Dengue, Zika or Chikungunya antigen, removing Zika false positives; and
(e) at least one fluid flow pathway capable of detecting a IgM or IgG antibody that can bind to any one of Dengue, Zika or Chikungunya antigen, removing Chikungunya false positives.

17. The device of claim 1, wherein the detection of the analyte by the first and/or second readout bands indicates whether a patient has an active or past infection, a cancer/tumor, an allergy/allergic immune response, or a non-communicable disease.

18. The device of claim 17, wherein (a) the infection is selected from a Mosquito borne illnesses, including but are not limited to malaria, dengue fever, West Nile virus, chikungunya, yellow fever, filariasis, Japanese encephalitis, Saint Louis encephalitis, Western equine encephalitis, Eastern equine encephalitis, Venezuelan equine encephalitis, La Crosse encephalitis and Zika fever;
(b) the tumor antigen is selected from MAGE-A1, MAGE-A2, MAGE-A3, MAGE-A4, MAGE-A5, MAGE-A6, MAGE-A7, MAGE-A8, MAGE-A9,MAGE-A10, MAGE-A11, MAGE-A12, GAGE-1, GAGE-2, GAGE-3, GAGE-4, GAGE-5, GAGE-6, GAGE-7, GAGE-8, GAGE-9, RAGE, BAGE-1, RAGE, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2,MAGE-C3, MAGE-C4, MAGE-C5, NY-ESO-I, LAGE-I, SSX-1, SSX-2 (1-HOM-MEL-40), SSX-3, SSX-4, SSX-5, SCP-I and XAGE, melanocyte differentiation antigens, p53, ras, p21ras, CEA, MUC1, PMSA, PSA, tyrosinase, Melan-A, MART-1, gp100, gp75, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-2, and 3, neo-PAP, myosin class I, OS-9, pml-RAR alpha fusion protein, PTPRK, K-ras, N-ras, Triosephosphate isomerase, GnT-V, Herv-K-mel, NA-88, SP17, and TRP2-Int2, MART-1, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, alpha.-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\170K, NY-CO-1, RCAS1, SDCCAG16, TA-90 (Mac-2 binding protein\cyclophilin C-associated protein), TAAL6, TAG72, TLP, TPS, tyrosinase related proteins, TRP-1, TRP-2, TRP-2/INT2, or mesothelin, HER-2/neu, 707-A, AFP, ART-4, p190 minor bcr-ab1, CAMEL, CAP-1, CAP-2, CDC27, CT, Cyp-B, DAM, ELF2M, GAGE, RAGE, HLA-A*0201, HLA-A*1101, HLA-A*0201-R1701, HLA-B*0702, HPV-E7, HAST-2, hTERT (or hTRT), iCE, LAGE, LDLR/FUT, MC1R, myosine/m, MUC1, NA88-A, NY-ESO-1, PRAME, PSA-1, PSA-2, PSA-3, prostate-specific membrane antigen (PSMA), PSM, RU1 or RU2, SAGE, SART-1 or SART-3, TEL/AML1, TPI/m, and WT1, adenosine deaminase-binding protein (ADAbp), FAP, cyclophlin b, CRC C017-1A/GA733, AML1, CD20, alpha-fetoprotein, E-cadherin, alpha-catenin, beta-catenin, gamma-catenin, p120ctn, adenomatous polyposis coli protein (APC), fodrin, Connexin 37, Ig-idiotype, p15, GM2 ganglioside, GD2 ganglioside, Smad family of tumor antigens, Imp-1, PIA, EBV-encoded nuclear antigen (EBNA)-1, brain glycogen phosphorylase, CT-7, and c-erhB-2;
(c) wherein infection is selected from a tick-borne illness, including but not limited to Anaplasmosis, Babesiosis, Borrelia mayonii, Borrelia miyamotoi, Colorado tick fever, Ehrlichiosis, Heartland virus, Lyme disease, Powassan disease, Rickettsia parkeri rickettsiosis, Rocky mountain spotted fever (RMSF), Southern tick-associated rash illness (START), Tickborne relapsing fever (TBRF), Tularemia, and 364D rickettsiosis;
(d) wherein the non-communicable disease is selected from to cardiovascular disease (e.g., coronary heart disease, stroke, etc.), cancer, chronic respiratory disease, diabetes, chronic neurologic disorders (e.g., Alzheimer's disease, dementias), and arthritis/musculoskeletal diseases;
(e) wherein the infection is selected from a bacterial illness, including but not limited to Streptococcal bacteria, Escherichia coli, Salmonella, Helicobacter pylori, Neisseria gonorrhoeae, Neisseria meningitides, Staphylococcus aureus, Streptococcal bacteria, Actinomycoses and nocardiosis, Anthrax, Brucellosis, Buruli ulcer, Capnocytophaga, Elizabethkingia, Glanders (Burkholderia mallei), Hansen's disease (Leprosy), Leptospirosis, Melioidosis (Burkholderia pseudomallei), Pasteurella sp. infections, and Rat-Bite fever;
(f) wherein the infection is selected from a parasitic illness, including but not limited to protozoan (e.g., leishmaniasis, chagas disease, malaria, toxoplasmosis, etc.) and helminths (e.g., tapeworms, flukes, roundworms, etc.);
(g) wherein the infection is selected from a fungal illness, including but not limited to Aspergillosis, Blastomycosis, Candidiasis, Coccidioidomycosis, C. neoformans Infection, C. gattii Infection, Fungal Eye Infections, Fungal Nail Infections, Histoplasmosis, Mucormycosis, Pneumocystis pneumonia, Ringworm, and Sporotrichosis;
(h) wherein the allergy is selected from a food preferably, peanuts, tree nuts, crustacean shellfish, fish, wheat (gluten), milk, egg, soybeans and/or airborne allergies, preferably pollen, mold, dust mite, and/or animals;
(i) wherein the infection is HIV; and/or
(j) the cancer is a leukemia, adenocarcinoma, sarcoma, skin cancer, melanoma, bladder cancer, brain cancer, breast cancer, uterine cancer, ovarian cancer, prostate cancer, lung cancer, colorectal cancer, cervical cancer, liver cancer, head and neck cancer, esophageal cancer, pancreas cancer, pancreatic ductal adenocarcinoma (PDA), renal cancer, stomach cancer, multiple myeloma and/or cerebral cancer.

19. Use of the device of claim 1 to detect whether a patient has an active or past infection, a cancer/tumor, an allergy/allergic immune response, or a non-communicable disease.

20. The use of claim 19, wherein the device is used to determine whether the patient has an infection selected from Dengue, Zika, Chikungunya, or HIV, a disease such as malaria, a cancer such as a leukemia, or an allergy such as: food allergies, preferably, peanuts, tree nuts, crustacean shellfish, fish, wheat (gluten), milk, egg, soybeans and/or airborne allergies, preferably pollen, mold, dust mite, and/or animals.
